SEQUENCE LISTING (43 sequences)

SEQ ID NO: 1 - protein, 321 aa, synthetic construct
MSEQSKDLSDPNFAAEAPNSEVHSSPGVSEGVPPSATLAEPQSPPLGPTAAPQAAPPPQAPNDEGDPKALQQAAEEGRAHQAPSAAQPGPAPPAPAQLVQKAHELMWYVLVKDQKKMIIWFPDMVKDVIGSYKKWCRSILRRTSLILARVFGLHLRLTSLHTMEFALVKALEPEELDRVALSNRMPMTGLLLMILSLIYVKGRGARESAVWNVLRILGLRPWKKHSTFGDVRKLITEEFVQMNYLKYQRVPYVEPPEYEFFWGSRASREITKMQIMEFLARVFKKDPQAWPSRYREALEEARALREANPTAHYPRSSVSED

SEQ ID NO: 2 - protein, 71 aa, synthetic construct
MERARDRLHLRRTTEQHVPEVEVQVKRRRTASLSNQECQLYPRRSQQQQVPVVDFQAELRQAFLAETPRGG

SEQ ID NO: 3 - protein, 240 aa, synthetic construct [sequence omitted]

SEQ ID NO: 4 - RNA, 95 nt, synthetic construct
  source 1..95: mol_type = other RNA
gccttggctaggttcatgatgacacaggaccttgtctgaacataatgatttcaaaatttgagcttaaaaatgacactctgaaatccagtcagtgt

SEQ ID NO: 5 - RNA, 77 nt, synthetic construct
  source 1..77: mol_type = other RNA
gggctggatttgtgatgagctgtgtttactgagcatgatgaagtaaagctcaacgtgattactctgaagtccagcct

SEQ ID NO: 6 - RNA, 108 nt, synthetic construct
  source 1..108: mol_type = other RNA
tgtgcttggatcgatgatgagtcccccataaaaacattccttggaaaagctgaacaaaatgagtgagaactcataccgtcgttctcatcggaactgaggtccagcaca

SEQ ID NO: 7 - RNA, 105 nt, synthetic construct
  source 1..105: mol_type = other RNA
tgtgcatggatcgatgatgacttccatatatacattccttggaaagctgaacaaaatgagtgaaaactctataccgtcatcctcgtcgaactgaggtccagcaca

SEQ ID NO: 8 - RNA, 103 nt, synthetic construct
  source 1..103: mol_type = other RNA
tgccctgggttgggtcaatgatgagaaccttatattgtcctgaagagaggtgatgacttaaaaatcatgctcaataggattacgctgaggcccagcctaggtg

SEQ ID NO: 9 - RNA, 85 nt, synthetic construct
  source 1..85: mol_type = other RNA
tgcttgtttggatcgatgatgagaataattgtctgaggatgctgagggactcattccagatgtcaatctgaggtccagatgtgtg

SEQ ID NO: 10 - DNA, 12 bp, synthetic construct
  source 1..12: mol_type = other DNA
ygcgcangcgcr

SEQ ID NO: 11 - DNA, 12 bp, synthetic construct
  source 1..12: mol_type = other DNA
cgcgcatgcgca

SEQ ID NO: 12 - DNA, 12 bp, synthetic construct
  source 1..12: mol_type = other DNA
gtcgcatgcgca

SEQ ID NO: 13 - DNA, 12 bp, synthetic construct
  source 1..12: mol_type = other DNA
cgcgcaggcgcg

SEQ ID NO: 14 - DNA, 12 bp, synthetic construct
  source 1..12: mol_type = other DNA
tgcgcatgcgcg

SEQ ID NO: 15 - DNA, 12 bp, synthetic construct
  source 1..12: mol_type = other DNA
tgcgcaagcgcg

SEQ ID NO: 16 - DNA, 12 bp, synthetic construct
  source 1..12: mol_type = other DNA
tgcgcatgcgca

SEQ ID NO: 17 - DNA, 12 bp, synthetic construct
  source 1..12: mol_type = other DNA
ggcgcctgcgca

SEQ ID NO: 18 - DNA, 12 bp, synthetic construct
  source 1..12: mol_type = other DNA
cgcgcaggcgca

SEQ ID NO: 19 - DNA, 12 bp, synthetic construct
  source 1..12: mol_type = other DNA
tgcgcaggcgtg

SEQ ID NO: 20 - DNA, 12 bp, synthetic construct
  source 1..12: mol_type = other DNA
ggcgcatgcgca

SEQ ID NO: 21 - DNA, 10 bp, synthetic construct
  source 1..10: mol_type = other DNA
cctctgggcc

SEQ ID NO: 22 - DNA, 10 bp, synthetic construct
  source 1..10: mol_type = other DNA
ccagtctagc

SEQ ID NO: 23 - DNA, 10 bp, synthetic construct
  source 1..10: mol_type = other DNA
ctagtcaggc

SEQ ID NO: 24 - DNA, 10 bp, synthetic construct
  source 1..10: mol_type = other DNA
ccattgcctg

SEQ ID NO: 25 - DNA, 10 bp, synthetic construct
  source 1..10: mol_type = other DNA
ctactcgcac

SEQ ID NO: 26 - DNA, 10 bp, synthetic construct
  source 1..10: mol_type = other DNA
cctttgcatg

SEQ ID NO: 27 - DNA, 10 bp, synthetic construct
  source 1..10: mol_type = other DNA
caatggtctg

SEQ ID NO: 28 - DNA, 10 bp, synthetic construct
  source 1..10: mol_type = other DNA
cacgtcagac

SEQ ID NO: 29 - DNA, 10 bp, synthetic construct
  source 1..10: mol_type = other DNA
cacgtcgggc

SEQ ID NO: 30 - DNA, 78 bp, synthetic construct
  source 1..78: mol_type = other DNA
cgcgcatgcgcacctctgggccgtcgcatgcgcaccagtctagccgcgcaggcgcgctagtcaggctgcgcatgcgcg

SEQ ID NO: 31 - DNA, 210 bp, synthetic construct
  source 1..210: mol_type = other DNA
cgcgcatgcgcacctctgggccgtcgcatgcgcaccagtctagccgcgcaggcgcgctagtcaggctgcgcatgcgcgccattgcctgtgcgcaagcgcgctactcgcactgcgcatgcgcacctttgcatgggcgcctgcgcacaatggtctgcgcgcaggcgcacacgtcagactgcgcaggcgtgcacgtcgggcggcgcatgcgca

SEQ ID NO: 32 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 33 - DNA, 141 bp, synthetic construct
  source 1..141: mol_type = other DNA
ggacgtccgtcgacgcgcgagcgagcgagtgactccggcgggcccgtttcgggcccgcagcccgctggaaaccagcgggccggagtcactcgctcgctcgcgcgtctctccctcaccggttgaggtagtgatccccaagga

SEQ ID NO: 34 - DNA, 141 bp, synthetic construct
  source 1..141: mol_type = other DNA
aggaacccctagtgatggagttggccactccctctctgcgcgctcgctcgctcactgaggccgggcgaccaaaggttgcccgacgcccgggctttgcccgggcggcctcagtgagcgagcgagcgcgcagctgcctgcagg

SEQ ID NO: 35 - DNA, 141 bp, synthetic construct
  source 1..141: mol_type = other DNA
tccttggggatcactacctcaaccggtgagggagagacgcgcgagcgagcgagtgactccggcccgctggtttccaacgggctgcgggcccgaaacgggcccgccggagtcactcgctcgctcgcgcgtcgacggacgtcc

SEQ ID NO: 36 - DNA, 1468 bp, synthetic construct
  source 1..1468: mol_type = other DNA
gcagagtggagcggccgccggagatgcctgacgcatctgtctgaggagcggtcagtgacgcgatggagcgggcaagggatcgcttacacctgagacgaactacagaacagcacgtaccagaggtggaagtccaagtcaaacgcagaaggactgcctcactgagcaaccaagagtgtcagttgtacccgaggcgttctcagcagcagcaagtacctgtggtggatttccaggctgaactgaggcaggcattcttagctgagacaccaagaggtggttaaagccatattggagtagcgaggaatctgattccaagcaaaaaccaggctccatctactctttgaagcttctgcccagcttgcattgtttctaggagaacctgcgtcatacctttatctatagccttcccctaggtcttcagaagcatcaagttttaactgtggacattggatttggtggaacagcaatcatgactgttggcaagagtagcaagatgctgcagcacattgactatagaatgagatgtatcctgcaagatggccgaatcttcattggcacctttaaggcttttgacaagcatatgaatttgatcctctgtgattgtgatgagttcagaaagatcaagccaaagaatgcgaagcaaccagagcgtgaagaaaagcgggttttgggtctggtgttgctgcgtggggagaacttggtatccatgactgtggaggggccaccccccaaagatactggcattgctcgggtaccacttgctggagctgctggaggccctggggttggtagggcagctggtagaggagtaccagctggtgtgccaattccccaggcccctgctggattggcaggccctgtccgaggagttgggggaccatcccagcaggtaatgactccacagggaagaggcactgtagcagctgctgctgttgctgcgactgccagtattgctggagccccaacacagtacccaccaggacggggcactccgcccccacccgtcggcagagcaaccccacctccaggcattatggctcctccacctggtatgagaccacccatgggcccaccaattgggcttccccctgctcgagggacgccaataggcatgccgcctccgggaatgagaccccctccaccaggcattagaggtccacctcccccaggaatgcgtccaccaagaccttagcatactgttgatccatctcagtcactttttcccctgcaatgcgtcttgtgaaattgtgtagagtgtttgtgagctttttgttccctcattctgcattaataatagctaataataaatgcatagagcaattaaactgtgaggtactgttgtatatatttttttgcctgttgattttgatgagatcttaagttactgtggatgagggtgatgcctattaagcagttgattcaaatcatattctctttaattcttaggataaaaaggttttctgctatctaa

SEQ ID NO: 37 - DNA, 95 bp, synthetic construct
  source 1..95: mol_type = other DNA
gccttggctaggttcatgatgacacaggaccttgtctgaacataatgatttcaaaatttgagcttaaaaatgacactctgaaatccagtcagtgt

SEQ ID NO: 38 - DNA, 77 bp, synthetic construct
  source 1..77: mol_type = other DNA
gggctggatttgtgatgagctgtgtttactgagcatgatgaagtaaagctcaacgtgattactctgaagtccagcct

SEQ ID NO: 39 - DNA, 108 bp, synthetic construct
  source 1..108: mol_type = other DNA
tgtgcttggatcgatgatgagtcccccataaaaacattccttggaaaagctgaacaaaatgagtgagaactcataccgtcgttctcatcggaactgaggtccagcaca

SEQ ID NO: 40 - DNA, 105 bp, synthetic construct
  source 1..105: mol_type = other DNA
tgtgcatggatcgatgatgacttccatatatacattccttggaaagctgaacaaaatgagtgaaaactctataccgtcatcctcgtcgaactgaggtccagcaca

SEQ ID NO: 41 - DNA, 103 bp, synthetic construct
  source 1..103: mol_type = other DNA
tgccctgggttgggtcaatgatgagaaccttatattgtcctgaagagaggtgatgacttaaaaatcatgctcaataggattacgctgaggcccagcctaggtg

SEQ ID NO: 42 - DNA, 85 bp, synthetic construct
  source 1..85: mol_type = other DNA
tgcttgtttggatcgatgatgagaataattgtctgaggatgctgagggactcattccagatgtcaatctgaggtccagatgtgtg

SEQ ID NO: 43 - DNA, 1906 bp, Homo sapiens
  source 1..1906: mol_type = genomic DNA
gcacttcctctccaggaatccgcggagggagcgcaggctcgaagagctcctggacgcagaggccctgcccttgccagacggcgcagacatgtcagaacaaagtaaggatctgagcgaccctaactttgcagccgaggcccccaactccgaggtgcacagcagccctggggtttcggagggggttcctccgtccgcgaccctggcagagccgcagagccctcctctaggcccgacggccgctccgcaggccgcgccgcctccccaggccccgaacgacgagggcgacccgaaggccctgcagcaggctgcggaggagggccgcgcccaccaggccccgagcgcggcccagccgggcccggcaccgccagccccggcgcagctggtgcagaaggcgcacgagctcatgtggtacgtgctggtcaaggaccagaagaagatgatcatctggtttccagacatggtgaaagatgtcatcggcagctacaagaagtggtgcaggagcatcctccggcgcaccagcctcatcctcgcccgggtgttcgggctgcacctgaggctaaccagcctgcacaccatggagtttgcgctggtcaaagcgctggagcccgaggagctggacagggtggcgctgagcaaccgcatgcccatgacaggcctcctgctcatgatcctgagcctcatctacgtgaagggccgcggcgccagagagagcgccgtctggaacgtgctgcgcatcctggggctgcggccctggaagaagcactccaccttcggggacgtgcggaagctcatcactgaggagttcgtccaaatgaattacctgaagtaccagcgcgtcccatacgtggagccgcccgaatacgagttcttttggggctcccgggccagccgcgaaatcaccaagatgcaaatcatggagttcctggccagggtctttaagaaagacccccaggcctggccctcccgatacagagaagctctggaggaggccagagctctgcgggaggctaatcccactgcccactaccctcgcagcagtgtctctgaggactagcaaagtctggaggcagatgaatggtttctgaccctcaccagggctgtggaagggtgggggtgggtcattatagtattcaggatttacagtgcagtattcacgtgtaacttttaagttttcagtacagtgcttttatacctttaatgcaatgttgtattcatttgggtactattgtgtagtatttaggatgtatgcatgtttgtttatatgtaagcttggttggtgctttcgcttttgtgctacctttcttggatttttgtaccagagatgtgctaaactgatgaaatacattgagaaagtttccatcttattcttttatatgggactgatgatgtgtgttggggtagactgctcctgcagagtttggaagaagtcaccagcaaagccggcctaaccaagaaaagtcaaggcccttcatgaccttgctgggcacagaaaacaccctcgtggagtacactaatttgaactggactggtctcagtgtgagcacttggcacactttactaaacacatatacaaccccaccgtgagtcaactttaaagtaaacattaaagattcttgtgatacaatcatttttggaaaagtgtactttatcattttaacaaagcagtatggttgggaatgagacaattctctattttacagtgtatacagatacaactatttcccctaatagggtgggaaaaatcgctactcatgattactcctaaatttgtgaagtttatagttctattgtctttaaatgtaactcatgtttatttcaaaaacattcacaaatatagaaaagtatacaaaacaaaacagtaagattgtctgtaatcacatcatatgggaataaaaaacaaaaataa

(19) United States
(12) Patent Application Publication
(10) Pub. No.: US 2026/0248959 A1
(43) Pub. Date: Aug. 27, 2026

(54) METHODS AND MATERIALS FOR TREATING PRADER-WILLI SYNDROME

(71) Applicant: UNIVERSITY OF PITTSBURGH - OF THE COMMONWEALTH SYSTEM OF HIGHER EDUCATION, Pittsburgh, PA (US)

(72) Inventor: Robert David Nicholls, Pittsburgh, PA (US)

(21) Appl. No.: 19/160,188

(22) PCT Filed: Feb. 27, 2024
(86) PCT No.: PCT/US2024/017458

§ 371 Date: Aug. 27, 2025

Related U.S. Application Data
(60) Provisional application No. 63/448,587, filed on Feb. 27, 2023.

(51) Int. Cl. A61K 48/00; A61K 38/17; A61P 43/00; C07K 14/47; C12N 15/86

(52) U.S. Cl. CPC A61K 48/0033; A61K 38/1709; A61K 48/005; A61P 43/00; C07K 14/47; C12N 15/86; C12N 2750/14143; C12N 2830/50

(57) ABSTRACT

This document provides methods and materials for treating a mammal (e.g., a human) having Prader-Willi syndrome (PWS). For example, adeno-associated virus (AAV) vectors can be designed to express one or more nucleic acids associated with PWS (PWS nucleic acids) and administered to a mammal (e.g., a human) having PWS to treat the mammal.

### CROSS-REFERENCE TO RELATED APPLICATIONS

This application claims the benefit of U.S. Patent Application Ser. No. 63/448,587, filed on Feb. 27, 2023. The disclosure of the prior application is considered part of, and is incorporated by reference in, the disclosure of this application.

### SEQUENCE LISTING

This application contains a Sequence Listing that has been submitted electronically as an XML file named “48881-0052WO1_SL.xml.” The XML file, created on Feb. 13, 2024, is 44000 bytes in size. The material in the XML file is hereby incorporated by reference in its entirety.

### TECHNICAL FIELD

This document relates to methods and materials for treating a mammal (e.g., a human) having Prader-Willi syndrome (PWS). For example, adeno-associated virus (AAV) vectors can be designed to express one or more nucleic acids associated with PWS (PWS nucleic acids) and administered to a mammal (e.g., a human) having PWS to treat the mammal.

### BACKGROUND

PWS is a neurodevelopmental disorder, characterized by numerous behavioral abnormalities and negative impacts on life expectancy and/or life quality. PWS is genetically complex with loss of function of a 2-3 Mb domain of approximately 10 paternally-expressed, imprinted genes (Nicholls et al., Annu. Rev. Genomics Hum. Genet., 2:153-175 (2001); Chamberlain et al., Neurobiol. Dis., 39:13-20 (2010); and Chung et al., Open Biol., 10:200195 (2020)), all with different and poorly understood, or even unknown, functions. Despite advances in understanding genetic causes of PWS and having animal models that mimic some clinical components, the actual causes of PWS clinical features are not known. Consequently, current therapeutic approaches for PWS utilize behavioral modification, such as change in diet and growth hormone (GH)-treatment. Although such therapeutic approaches can improve outcomes, none is a cure, and significant clinical morbidity persists (Chung et al., Open Biol., 10:200195 (2020); and Tauber et al., Lancet Diabetes Endocrinol., 9:235-246 (2021)). Development of new therapies to reactivate or replace silent or missing PWS genes is complicated by heterochromatic silencing or the large number of genes (Chung et al., Open Biol., 10:200195 (2020); Wang et al., Trends Pharmacol. Sci., 40:605-608 (2019); and Kim et al., Transl. Res., 208:105-118 (2019)).

### SUMMARY

This document provides methods and materials for treating a mammal (e.g., a human) having PWS. In some cases, this document provides AAV vectors designed to express one or more (e.g., one, two, three, four, five, six, seven, eight, or more) PWS nucleic acids. As described herein, AAV vectors can be designed to express one or more PWS nucleic acids and can be administered to a mammal (e.g., a human) having PWS to treat the mammal. For example, an AAV vector having a genome (e.g., a single-stranded or double-stranded DNA genome) including a nucleic acid sequence (e.g., an engineered DNA sequence) including (a) a nucleotide sequence including a nucleotide sequence encoding a necdin (NDN) polypeptide, (b) a nucleotide sequence encoding a SNRPN upstream reading frame (SNURF) polypeptide, (c) a nucleotide sequence including a nucleotide sequence encoding a small nuclear ribonucleoprotein-associated protein N (SNRPN) polypeptide, (d) a nucleotide sequence encoding a SNORD107 small nucleolar RNA (snoRNA), (e) a nucleotide sequence encoding a SNORD64 snoRNA, (f) a nucleotide sequence encoding a SNORD116 snoRNA, (g) a nucleotide sequence encoding a SNORD115 snoRNA, and (h) a nucleotide sequence encoding a SNORD109 snoRNA can increase expression of a NDN polypeptide, a SNURF polypeptide, a SNRPN polypeptide, a SNORD107 snoRNA, a SNORD64 snoRNA, a SNORD116 snoRNA, a SNORD115 snoRNA, and/or a SNORD109 snoRNA within a cell (e.g., within a cell within a mammal such as a human).

Having the ability to increase expression of one or more PWS nucleic acids within a cell (e.g., within a cell within a mammal such as a human) using one or more AAV vectors provided herein (e.g., a population of a single AAV vector having a genome including a nucleic acid sequence (e.g., an engineered DNA sequence) including (a) a nucleotide sequence including a nucleotide sequence encoding a NDN polypeptide, (b) a nucleotide sequence encoding a SNURF polypeptide, (c) a nucleotide sequence including a nucleotide sequence encoding a SNRPN polypeptide, (d) a nucleotide sequence encoding a SNORD107 sequence encoding a SNORD116 snoRNA, (g) a nucleotide sequence encoding a SNORD115 snoRNA, and (h) a nucleotide sequence encoding a SNORD109 snoRNA) provides a unique and unrealized opportunity to treat having PWS. In addition, AAV vectors provided herein (e.g., one or more AAV vectors designed to express one or more PWS nucleic acids) can be produced using cost-effective methods. In some cases, a population of a single AAV vector provided herein can be used to increase expression of one or more PWS nucleic acids to treat PWS.

In general, one aspect of this document features adeno-associated viruses having a single-stranded DNA, where the single-stranded DNA comprises a first inverted terminal repeat (ITR) sequence followed by an engineered DNA sequence followed by a second ITR sequence, where the engineered DNA sequence comprises (a) a first DNA sequence comprising a first promotor sequence operably linked to first DNA segment comprising a nucleotide sequence encoding a necdin polypeptide and (b) a second DNA sequence comprising a second promotor sequence operably linked to a second DNA segment comprising a nucleotide sequence encoding a SNURF polypeptide, a nucleotide sequence encoding a SNRPN polypeptide, a nucleotide sequence encoding a SNORD107 small nucleolar RNA (snoRNA), a nucleotide sequence encoding a SNORD64 snoRNA, a nucleotide sequence encoding a SNORD116 snoRNA, a nucleotide sequence encoding a SNORD 115 snoRNA, and a nucleotide sequence encoding a SNORD109 snoRNA, where each of the first promotor sequence and the second promotor sequence comprises at least four motifs having the DNA sequence set forth in SEQ ID NO: 10 (5′-YGCGCANGCGCR-3′), where 8 to 12 intervening nucleotides are located between each adjacent motif of the at least four motifs, and where delivery of the adeno-associated virus to a cell within the mammal results in expression of the necdin polypeptide, the SNURF polypeptide, the SNRPN polypeptide, the SNORD107 snoRNA, the SNORD64 snoRNA, the SNORD116 snoRNA, the SNORD 115 snoRNA, and the SNORD 109 snoRNA. The mammal can be a human. The cell can be a pancreatic islet endocrine cell. The cell can be a gut endocrine cell. The cell can be a pituitary neuroendocrine cell. The adeno-associated virus can be AAV6, AAV8, AAV3, AAVDJ, AAVKP1, or AAV9. The first ITR sequence can be directly followed by the engineered DNA sequence. The first ITR sequence can be indirectly followed by the engineered DNA sequence. The engineered DNA sequence can be directly followed by the second ITR sequence. The engineered DNA sequence can be indirectly followed by the second ITR sequence. The first ITR sequence can include the DNA sequence set forth in SEQ ID NO:32 and the first ITR sequence can include the DNA sequence set forth in SEQ ID NO: 34. The first promotor sequence can include four of the motifs. The second promotor sequence can include six of the motifs. Each of the motifs can include a DNA sequence set forth in any one of SEQ ID NOs: 11 to 20. Each of the motifs can include a DNA sequence set forth in any one of SEQ ID NOs: 11 to 14. Each of the 8 to 12 intervening nucleotides can include a DNA sequence set forth in any one of SEQ ID NOs: 21 to 29. Each of the 8 to 12 intervening nucleotides can include a DNA sequence set forth in any one of SEQ ID NOs: 21 to 23. The necdin polypeptide can be a human necdin polypeptide. The necdin polypeptide can include the amino acid sequence set forth in SEQ ID NO:1. The SNURF polypeptide can be a human SNURF polypeptide. The SNURF polypeptide can include the amino acid sequence set forth in SEQ ID NO:2. The SNRPN polypeptide can be a human SNRPN polypeptide. The SNRPN polypeptide can include the amino acid sequence set forth in SEQ ID NO: 3. The SNORD107 snoRNA can be a human SNORD107 snoRNA. The SNORD107 snoRNA can include the RNA sequence set forth in SEQ ID NO:4. The SNORD64 snoRNA can be a human SNORD64 snoRNA. The SNORD64 snoRNA can include the RNA sequence set forth in SEQ ID NO:5. The SNORD116 snoRNA can be a human SNORD116 snoRNA. The SNORD116 snoRNA can include the RNA sequence set forth in SEQ ID NO:6 or SEQ ID NO:7. The SNORD115 snoRNA can be a human SNORD115 snoRNA. The SNORD 115 snoRNA can include the RNA sequence set forth in SEQ ID NO:8. The SNORD 109 snoRNA can be a human SNORD 109 snoRNA. The SNORD 109 snoRNA can include the RNA sequence set forth in SEQ ID NO:9. The first DNA segment can include, downstream of the nucleotide sequence encoding the necdin polypeptide, a poly (A) signal. The poly (A) signal can include an SV40 poly (A) signal. The poly (A) signal can include the DNA sequence AATAAA or the DNA sequence ATTAAA. The second DNA segment can include, downstream of the nucleotide sequence encoding the SNURF polypeptide, the nucleotide sequence encoding the SNRPN polypeptide, the nucleotide sequence encoding the SNORD 107 snoRNA, the nucleotide sequence encoding the SNORD64 snoRNA, the nucleotide sequence encoding the SNORD116 snoRNA, the nucleotide sequence encoding the SNORD 115 snoRNA, and the nucleotide sequence encoding the SNORD109 snoRNA, a poly (A) signal. The poly (A) signal can include an SV40 poly (A) signal. The poly (A) signal can include the DNA sequence AATAAA or the DNA sequence ATTAAA.

In another aspect, this document features isolated nucleic acid molecules comprising the engineered DNA sequence having (a) a first DNA sequence comprising a first promotor sequence operably linked to first DNA segment comprising a nucleotide sequence encoding a necdin polypeptide and (b) a second DNA sequence comprising a second promotor sequence operably linked to a second DNA segment comprising a nucleotide sequence encoding a SNURF polypeptide, a nucleotide sequence encoding a SNRPN polypeptide, a nucleotide sequence encoding a SNORD107 small nucleolar RNA (snoRNA), a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA, where each of the first promotor sequence and the second promotor sequence comprises at least four motifs having the DNA sequence set forth in SEQ ID NO:10 (5′-YGCGCANGCGCR-3′), where 8 to 12 intervening nucleotides are located between each adjacent motif of the at least four motifs, and where delivery of the adeno-associated virus to a cell within the mammal results in expression of the necdin polypeptide, the SNURF polypeptide, the SNRPN polypeptide, the SNORD107 snoRNA, the SNORD64 snoRNA, the SNORD 116 snoRNA, the SNORD 115 snoRNA, and the SNORD109 snoRNA.

In another aspect, this document features in vitro host cells, where the host cell comprises an adeno-associated virus having a single-stranded DNA, where the single-stranded DNA comprises a first ITR sequence followed by an engineered DNA sequence followed by a second ITR sequence, where the engineered DNA sequence comprises (a) a first DNA sequence comprising a first promotor sequence operably linked to first DNA segment comprising a nucleotide sequence encoding a necdin polypeptide and (b) a second DNA sequence comprising a second promotor sequence operably linked to a second DNA segment comprising a nucleotide sequence encoding a SNURF polypeptide, a nucleotide sequence encoding a SNRPN polypeptide, a nucleotide sequence encoding a SNORD107 small nucleolar RNA (snoRNA), a nucleotide sequence encoding a SNORD64 snoRNA, a nucleotide sequence encoding a SNORD116 snoRNA, a nucleotide sequence encoding a SNORD 115 snoRNA, and a nucleotide sequence encoding a SNORD109 snoRNA, where each of the first promotor sequence and the second promotor sequence comprises at least four motifs having the DNA sequence set forth in SEQ ID NO:10 (5′-YGCGCANGCGCR-3′), where 8 to 12 intervening nucleotides are located between each adjacent motif of the at least four motifs, and where delivery of the adeno-associated virus to a cell within the mammal in results in expression of the necdin polypeptide, the SNURF polypeptide, the SNRPN polypeptide, the SNORD107 snoRNA, the SNORD64 snoRNA, the SNORD116 snoRNA, the SNORD115 snoRNA, and the SNORD 109 snoRNA. The host cell can be an epithelial cell.

In another aspect, this document features in vitro host cells, where the host cell comprises a nucleic acid having a (a) a first DNA sequence comprising a first promotor sequence operably linked to first DNA segment comprising a nucleotide sequence encoding a necdin polypeptide and (b) a second DNA sequence comprising a second promotor sequence operably linked to a second DNA segment comprising a nucleotide sequence encoding a SNURF polypeptide, a nucleotide sequence encoding a SNRPN polypeptide, a nucleotide sequence encoding a SNORD107 small nucleolar RNA (snoRNA), a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA, where each of the first promotor sequence and the second promotor sequence comprises at least four motifs having the DNA sequence set forth in SEQ ID NO:10 (5′-YGCGCANGCGCR-3′), where 8 to 12 intervening nucleotides are located between each adjacent motif of the at least four motifs, and where delivery of the adeno-associated virus to a cell within the mammal results in expression of the necdin polypeptide, the SNURF polypeptide, the SNRPN polypeptide, the SNORD107 snoRNA, the SNORD64 snoRNA, the SNORD 116 snoRNA, the SNORD115 snoRNA, and the SNORD 109 snoRNA. The host cell can be an epithelial cell.

In another aspect, this document features compositions comprising an adeno-associated virus having a single-stranded DNA, where the single-stranded DNA comprises a ITR sequence followed by an engineered DNA sequence followed by a second ITR sequence, where the engineered DNA sequence comprises (a) a first DNA sequence comprising a first promotor sequence operably linked to first DNA segment comprising a nucleotide sequence encoding a necdin polypeptide and (b) a second DNA sequence comprising a second promotor sequence operably linked to a second DNA segment comprising a nucleotide sequence encoding a SNURF polypeptide, a nucleotide sequence encoding a SNRPN polypeptide, a nucleotide sequence encoding a SNORD107 small nucleolar RNA (snoRNA), a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA, where each of the first promotor sequence and the second promotor sequence comprises at least four motifs having the DNA sequence set forth in SEQ ID NO: 10 (5′-YGCGCANGCGCR-3′), where 8 to 12 intervening nucleotides are located between each adjacent motif of the at least four motifs, and where delivery of the adeno-associated virus to a cell within the mammal results in expression of the necdin polypeptide, the SNURF polypeptide, the SNRPN polypeptide, the SNORD107 snoRNA, the SNORD64 snoRNA, the SNORD116 snoRNA, the SNORD115 snoRNA, and the SNORD109 snoRNA.

In another aspect, this document features methods for increasing expression of a necdin polypeptide, a SNURF polypeptide, a SNRPN polypeptide, a SNORD107 snoRNA, a SNORD64 snoRNA, a SNORD116 snoRNA, a SNORD115 snoRNA, and a SNORD109 snoRNA by cells within a mammal. The methods can include, or consist essentially of, administering an adeno-associated virus having a single-stranded DNA, where the single-stranded DNA comprises a first ITR sequence followed by an engineered DNA sequence followed by a second ITR sequence, where the engineered DNA sequence comprises (a) a first DNA sequence comprising a first promotor sequence operably linked to first DNA segment comprising a nucleotide sequence encoding a necdin polypeptide and (b) a second DNA sequence comprising a second promotor sequence operably linked to a second DNA segment comprising a nucleotide sequence encoding a SNURF polypeptide, a nucleotide sequence encoding a SNRPN polypeptide, a nucleotide sequence encoding a SNORD107 small nucleolar RNA (snoRNA), a nucleotide sequence encoding a SNORD64 snoRNA, a nucleotide sequence encoding a SNORD116 snoRNA, a nucleotide sequence encoding a SNORD 115 snoRNA, and a nucleotide sequence encoding a SNORD109 snoRNA, where each of the first promotor sequence and the second promotor sequence comprises at least four motifs having the DNA sequence set forth in SEQ ID NO:10 (5′-YGCGCANGCGCR-3′), where 8 to 12 intervening nucleotides are located between each adjacent motif of the at least four motifs, and where delivery of the adeno-associated virus to a cell within the mammal results in expression of the necdin polypeptide, the SNURF polypeptide, the SNRPN polypeptide, the SNORD107 snoRNA, the SNORD64 snoRNA, the SNORD116 snoRNA, the SNORD115 snoRNA, and the SNORD109 snoRNA to the mammal, thereby increasing expression of the necdin polypeptide, the SNURF polypeptide, the SNRPN polypeptide, the SNORD 107 snoRNA, the SNORD64 snoRNA, the SNORD116 snoRNA, the SNORD115 snoRNA, and the SNORD 109 snoRNA by the cells within the mammal. The mammal can be a human.

In another aspect, this document features methods for treating Prader-Willi syndrome. The methods can include, or consist essentially of, administering an adeno-associated virus having a single-stranded DNA, where the single-stranded DNA comprises a first ITR sequence followed by an engineered DNA sequence followed by a second ITR sequence, where the engineered DNA sequence comprises (a) a first DNA sequence comprising a first promotor sequence operably linked to first DNA segment comprising a nucleotide sequence encoding a necdin polypeptide and (b) a second DNA sequence comprising a second promotor sequence operably linked to a second DNA segment comprising a nucleotide sequence encoding a SNURF polypeptide, a nucleotide sequence encoding a SNRPN polypeptide, a nucleotide sequence encoding a SNORD107 small nucleolar RNA (snoRNA), a nucleotide sequence encoding a SNORD64 snoRNA, a nucleotide sequence encoding a SNORD 116 snoRNA, a nucleotide sequence encoding a SNORD115 snoRNA, and a nucleotide sequence encoding a SNORD109 snoRNA, where each of the first promotor sequence and the second promotor sequence comprises at least four motifs having the DNA sequence set forth in SEQ ID NO:10 (5′-YGCGCANGCGCR-3′), where 8 to 12 intervening nucleotides are located between each adjacent motif of the at least four motifs, and where delivery of the adeno-associated virus to a cell within the mammal results in expression of the necdin polypeptide, the SNURF polypeptide, the SNRPN polypeptide, the SNORD107 snoRNA, the SNORD64 snoRNA, the SNORD116 snoRNA, the SNORD115 snoRNA, and the SNORD 109 snoRNA to a mammal having Prader-Willi syndrome, where expression of a necdin polypeptide, a SNURF polypeptide, a SNRPN polypeptide, a SNORD107 snoRNA, a SNORD64 snoRNA, a SNORD 116 snoRNA, a SNORD115 snoRNA, and a SNORD109 snoRNA by cells within the mammal is increased following the administering step, and where severity of a symptom of the Prader-Willi syndrome is reduced following the administering step. The mammal can be a human.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A-1D. PWS-minigene design for single AAV delivery. FIG. 1A) Engineered DNA sequences including (a) a nucleotide sequence including a nucleotide sequence encoding a NDN polypeptide, (b) a nucleotide sequence encoding a SNURF polypeptide, (c) a nucleotide sequence including a nucleotide sequence encoding a SNRPN polypeptide, (d) a nucleotide sequence encoding a SNORD107 snoRNA, (e) a nucleotide sequence encoding a SNORD64 snoRNA, (f) a nucleotide sequence encoding a SNORD116 snoRNA, (g) a nucleotide sequence encoding a SNORD115 snoRNA, and (h) a nucleotide sequence encoding a SNORD109 snoRNA fit within the 4.75 kb packaging capacity of a single AAV vector. FIG. 1B) A schematic showing an exemplary design of a nucleotide sequence encoding a SNURF polypeptide, a SNRPN polypeptide, and five snoRNAs (i.e., a SNORD 107 snoRNA, a SNORD64 snoRNA, a SNORD116 snoRNA, a SNORD115 snoRNA, and a SNORD109 snoRNA), where the five snoRNAs are located within individual synthetic introns between protein-coding exons. FIG. 1C) A schematic showing an exemplary design of a nucleotide sequence encoding a SNURF polypeptide, a SNRPN polypeptide, and five snoRNAs (i.e., a SNORD107 snoRNA, a SNORD64 snoRNA, a SNORD 116 snoRNA, a SNORD115 snoRNA, and a SNORD109 snoRNA), where the five snoRNAs are clustered within a single segment between protein-coding exons. FIG. 1D) A schematic showing an exemplary design of a nucleotide sequence encoding a SNURF polypeptide, a SNRPN polypeptide, and five snoRNAs (i.e., a SNORD107 snoRNA, a SNORD64 snoRNA, a SNORD116 snoRNA, a SNORD115 snoRNA, and a SNORD109 snoRNA), where the five snoRNAs are located 3′ of SNURF-SNRPN as in the endogenous locus. Numbered boxes encode protein-coding exons of SNURF-SNRPN; NDN coding exon; snoRNAs are individually labeled (as is NDN protein-coding exon in FIG. 1A); unlabeled black boxes are non-coding rapidly degraded microexons (from SNHG1); the left most box is the 5′-UTRand the right most box is the 3′-UTR with polyA (pA) sites; in FIG. 1A, NRF1-binding sites in promoters are shown by hash marks.

FIG. 2. Expression levels of mNeonGreen in transfected INS-1 B-cells. mNeonGreen driven by the CMV promoter (left panel), by a 4×NRF1 promoter array (middle panel), or by a 10×NRF1 promoter array (right panel).

FIGS. 3A-3D. PWS INS-1 β-cell model. FIGS. 3A) Genome editing led to isolation of PWS-lines (3, 19-1, and 19-4) having 3.16 Mb deletions on the paternal allele with loss of expression of PWS nucleic acids (Snrpn, Snord116, and not shown), while control lines (2, 16, and 5-9+) expressed the PWS nucleic acids. All lines were clonally expanded from single cells. FIG. 3B) PWS β-cell lines have insulin secretion deficits at low (2.2 mM) and high (22 mM) glucose levels, based on the pooled insulin secretory rates from n=36 for each of PWS and control groups (n=12 per cell line). Although glucose-stimulated secretion increased 1.75-fold for control (p<0.0001) and 1.85-fold for PWS (p<0.0001), loss of expression of PWS genes decreased the secretory rate by 36% (p<0.0001) in basal 2.8 mM glucose conditions and by 32% (p<0.0001) in stimulatory 22 mM glucose conditions. FIGS. 3C and 3D) Reduced insulins and ER chaperones in PWS INSI β-cell lines. Insulin (FIG. 3C), mCherry, KDEL (FIG. 3D), and control (TUBG or GPI) antibodies were used in western blots of whole cell lysates from β-cell lines under control (DMSO) conditions for 5 hours. The insulin antibody detects all forms of insulin (PrePro, Pro, and fully processed), and mouse proINS2- or C-peptide (CP)-mCherry. The KDEL antibody detects GRP94 (endoplasmin) and GRP78 (BiP).

### DETAILED DESCRIPTION

This document provides methods and materials for treating a mammal (e.g., a human) having PWS. In some cases, this document provides AAV vectors designed to express one or more (e.g., one, two, three, four five, six, seven, eight, or more) PWS nucleic acids. For example, an AAV vector designed to express one or more PWS nucleic acids can have a genome (e.g., a single-stranded or double-stranded DNA genome) including (a) a nucleotide sequence including a nucleotide sequence encoding a NDN polypeptide, (b) a nucleotide sequence encoding a SNURF polypeptide, (c) a nucleotide sequence including a nucleotide sequence encoding a SNRPN polypeptide, (d) a nucleotide sequence encoding a SNORD107 sequence encoding a SNORD116 snoRNA, (g) a nucleotide sequence encoding a SNORD115 snoRNA, and (h) a nucleotide sequence encoding a SNORD109 snoRNA. In some cases, an AAV vector designed to express one or more PWS nucleic acids can have a genome (e.g., a single-stranded or double-stranded DNA genome) including a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA. In some cases, AAV vectors provided herein (e.g., a population of a single AAV vector designed to express one or more PWS nucleic acids) can increase expression of a NDN polypeptide, a SNURF polypeptide, a SNRPN polypeptide, a SNORD 107 snoRNA, a SNORD64 snoRNA, a SNORD116 snoRNA, a SNORD115 snoRNA, and/or a SNORD 109 snoRNA within a cell (e.g., within a cell within a mammal such as a human). In some cases, one or more AAV vectors provided herein (e.g., a population of a single AAV vector designed to express one or more PWS nucleic acids) can be administered to a mammal (e.g., a human) having PWS to treat the mammal. For example, a population of a single AAV vector provided herein (e.g., a population of a single AAV vector designed to express one or more PWS nucleic acids) can be used to increase expression of a NDN polypeptide, a SNURF polypeptide, a SNRPN polypeptide, a SNORD 107 snoRNA, a SNORD64 snoRNA, a SNORD116 snoRNA, a SNORD115 snoRNA, and/or a SNORD109 snoRNA by cells within a mammal (e.g., a human) having PWS to treat the mammal.

Any appropriate AAV can be designed to express one or more PWS nucleic acids (e.g., can be designed to have a genome including a nucleic acid sequence including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA). In some cases, an AAV vector designed to express one or more PWS nucleic acids can have a single-stranded or double-stranded DNA genome. When containing a single-stranded DNA genome, the AAV vector designed to express one or more PWS nucleic acids can be a positive-strand virus or a negative-strand virus. In some cases, an AAV vector designed to express one or more PWS nucleic acids can infect dividing cells. In some cases, an AAV vector designed to express one or more PWS nucleic acids can infect non-dividing cells. Examples AAV vectors that can be designed to express one or more PWS nucleic acids as described herein (e.g., designed to have a genome including a nucleic acid sequence including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA) include, without limitation, serotype 9 AAV (AAV9) vectors, AAV6 vectors, AAV8 vectors, AAV3 vectors, AAVDJ vectors, and AAVKP1 vectors.

An AAV vector provided herein (e.g., an AAV vector designed to express one or more PWS nucleic acids) can include any appropriate nucleic acid encoding a NDN polypeptide. For example, an AAV vector having a genome (e.g., a single-stranded or double-stranded DNA genome) including a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include any appropriate nucleic acid encoding a NDN polypeptide.

A nucleic acid encoding a NDN polypeptide in an AAV vector provided herein (e.g., a single AAV vector designed to express one or more PWS nucleic acids) can encode any appropriate NDN polypeptide. For example, an AAV vector having a genome (e.g., a single-stranded or double-stranded DNA genome) including a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include nucleic acid encoding any appropriate NDN polypeptide. In some cases, a NDN polypeptide that can be encoded by a nucleic acid encoding a NDN polypeptide in an AAV vector provided herein (e.g., an AAV vector designed to express one or more PWS nucleic acids) can have the amino acid sequence set forth in the National Center for Biotechnology Information (NCBI) databases at Accession No. NM 002487. In some cases, a NDN polypeptide that can be encoded by a nucleic acid encoding a NDN polypeptide in an AAV vector provided herein (e.g., an AAV vector designed to express one or more PWS nucleic acids) can have the amino acid sequence set forth in SEQ ID NO:1. In some cases, a NDN polypeptide that can be encoded by a nucleic acid encoding a NDN polypeptide in an AAV vector provided herein (e.g., an AAV vector designed to express one or more PWS nucleic acids) can comprise, consist essentially of, or consist of the amino acid sequence set forth in SEQ ID NO:1. In some cases, a NDN polypeptide that can be encoded by a nucleic acid encoding a NDN polypeptide in an AAV vector provided herein (e.g., an AAV vector designed to express one or more PWS nucleic acids) can comprise, consist essentially of, or consist of the amino acid sequence set forth in SEQ ID NO:1 with one, two, three, four, five, six, seven, eight, nine, or ten amino acid deletions, additions, substitutions, or combinations thereof. In some cases, a NDN polypeptide that can be encoded by a nucleic acid encoding a NDN polypeptide in an AAV vector provided herein (e.g., a single AAV vector designed to express one or more PWS nucleic acids) can comprise, consist essentially of, or consist of the amino acid sequence set forth in SEQ ID NO: 1 with two or less, three or less, four or less, five or less, six or less, seven or less, eight or less, nine or less, or ten or less amino acid deletions, additions, substitutions, or combinations thereof.

An AAV vector provided herein (e.g., an AAV vector designed to express one or more PWS nucleic acids) can include any appropriate nucleic acid encoding a SNURF polypeptide. For example, an AAV vector having a genome (e.g., a single-stranded or double-polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include any appropriate nucleic acid encoding a SNURF polypeptide.

A nucleic acid encoding a SNURF polypeptide in an AAV vector provided herein (e.g., a single AAV vector designed to express one or more PWS nucleic acids) can encode any appropriate SNURF polypeptide. For example, an AAV vector having a genome (e.g., a single-stranded or double-stranded DNA genome) including a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include nucleic acid encoding any appropriate SNURF polypeptide. In some cases, a SNURF polypeptide that can be encoded by a nucleic acid encoding a SNURF polypeptide in an AAV vector provided herein (e.g., an AAV vector designed to express one or more PWS nucleic acids) can have the amino acid sequence set forth in the NCBI databases at Accession No. NP_005669.2. In some cases, a SNURF polypeptide that can be encoded by a nucleic acid encoding a SNURF polypeptide in an AAV vector provided herein (e.g., an AAV vector designed to express one or more PWS nucleic acids) can have the amino acid sequence set forth in SEQ ID NO:2. In some cases, a SNURF polypeptide that can be encoded by a nucleic acid encoding a SNURF polypeptide in an AAV vector provided herein (e.g., an AAV vector designed to express one or more PWS nucleic acids) can comprise, consist essentially of, or consist of the amino acid sequence set forth in SEQ ID NO:2. In some cases, a SNURF polypeptide that can be encoded by a nucleic acid encoding a SNURF polypeptide in an AAV vector provided herein (e.g., an AAV vector designed to express one or more PWS nucleic acids) can comprise, consist essentially of, or consist of the amino acid sequence set forth in SEQ ID NO:2 with one, two, three, four, five, six, seven, eight, nine, or ten amino acid deletions, additions, substitutions, or combinations thereof. In some cases, a SNURF polypeptide that can be encoded by a nucleic acid encoding a SNURF polypeptide in an AAV vector provided herein (e.g., a single AAV vector designed to express one or more PWS nucleic acids) can comprise, consist essentially of, or consist of the amino acid sequence set forth in SEQ ID NO:2 with two or less, three or less, four or less, five or less, six or less, seven or less, eight or less, nine or less, or ten or less amino acid deletions, additions, substitutions, or combinations thereof.

An AAV vector provided herein (e.g., an AAV vector designed to express one or more PWS nucleic acids) can include any appropriate nucleic acid encoding a SNRPN polypeptide. For example, an AAV vector having a genome (e.g., a single-stranded or double-stranded DNA genome) including a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include any appropriate nucleic acid encoding a SNRPN polypeptide.

A nucleic acid encoding a SNRPN polypeptide in an AAV vector provided herein (e.g., a single AAV vector designed to express one or more PWS nucleic acids) can encode any appropriate SNRPN polypeptide. For example, an AAV vector having a genome (e.g., a single-stranded or double-stranded DNA genome) including a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include nucleic acid encoding any appropriate SNRPN polypeptide. In some cases, a SNRPN polypeptide that can be encoded by a nucleic acid encoding a SNRPN polypeptide in an AAV vector provided herein (e.g., an AAV vector designed to express one or more PWS nucleic acids) can have the amino acid sequence set forth in the NCBI databases at Accession No. NP_003088.1. In some cases, a SNRPN polypeptide that can be encoded by a nucleic acid encoding a SNRPN polypeptide in an AAV vector provided herein (e.g., an AAV vector designed to express one or more PWS nucleic acids) can have the amino acid sequence set forth in SEQ ID NO:3. In some cases, a SNRPN polypeptide that can be encoded by a nucleic acid encoding a SNRPN polypeptide in an AAV vector provided herein (e.g., an AAV vector designed to express one or more PWS nucleic acids) can comprise, consist essentially of, or consist of the amino acid sequence set forth in SEQ ID NO:3. In some cases, a SNRPN polypeptide that can be encoded by a nucleic acid encoding a SNRPN polypeptide in an AAV vector provided herein (e.g., an AAV vector designed to express one or more PWS nucleic acids) can comprise, consist essentially of, or consist of the amino acid sequence set forth in SEQ ID NO:3 with one, two, three, four, five, six, seven, eight, nine, or ten amino acid deletions, additions, substitutions, or combinations thereof. In some cases, a SNRPN polypeptide that can be encoded by a nucleic acid encoding a SNRPN polypeptide in an AAV vector provided herein (e.g., a single AAV vector designed to express one or more PWS nucleic acids) can comprise, consist essentially of, or consist of the amino acid sequence set forth in SEQ ID NO:3 with two or less, three or less, four or less, five or less, six or less, seven or less, eight or less, nine or less, or ten or less amino acid deletions, additions, substitutions, or combinations thereof.

An AAV vector provided herein (e.g., an AAV vector designed to express one or more PWS nucleic acids) can include a nucleic acid encoding any appropriate snoRNA. In some cases, a snoRNA can be a C/D box small nucleolar RNA (SNORD). Examples of snoRNAs that can be encoded by a nucleic acid encoding any appropriate snoRNA in an AAV vector provided herein (e.g., an AAV vector designed to express one or more PWS nucleic acids) include, without limitation, SNORD107 snoRNAs, SNORD64 snoRNAs, SNORD116 snoRNAs, SNORD 115 snoRNAs, and SNORD 109 snoRNAs (e.g., SNORD109A snoRNAs and SNORD109B snoRNAs).

In some cases, an AAV vector provided herein (e.g., an AAV vector designed to express one or more PWS nucleic acids) can include nucleic acid encoding a SNORD107 snoRNA. An AAV vector provided herein (e.g., an AAV vector designed to express one or more PWS nucleic acids) can include any appropriate nucleic acid encoding a SNORD107 snoRNA. For example, an AAV vector having a genome (e.g., a single-stranded or double-polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include any appropriate nucleotide sequence encoding a SNORD107 snoRNA.

A nucleic acid encoding a SNORD107 snoRNA in an AAV vector provided herein (e.g., a single AAV vector designed to express one or more PWS nucleic acids) can encode any appropriate SNORD107 snoRNA. For example, an AAV vector having a genome (e.g., a single-stranded or double-stranded DNA genome) including a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include nucleic acid encoding any appropriate SNORD107 snoRNA. In some cases, a SNORD 107 snoRNA that can be encoded by a nucleic acid encoding a SNORD107 snoRNA in an AAV vector provided herein (e.g., an AAV vector designed to express one or more PWS nucleic acids) can have the nucleic acid sequence set forth in the NCBI databases at Accession No. NR_001293. In some cases, a SNORD107 snoRNA that can be encoded by a nucleic acid encoding a SNORD107 snoRNA in an AAV vector provided herein (e.g., an AAV vector designed to express one or more PWS nucleic acids) can have the RNA sequence set forth in SEQ ID NO:4. In some cases, a SNORD107 snoRNA that can be encoded by a nucleic acid encoding a SNORD107 snoRNA in an AAV vector provided herein (e.g., an AAV vector designed to express one or more PWS nucleic acids can comprise, consist essentially of, or consist of the RNA sequence set forth in SEQ ID NO: 4. In some cases, a SNORD107 snoRNA that can be encoded by a nucleic acid encoding a SNORD107 snoRNA in an AAV vector provided herein (e.g., an AAV vector designed to express one or more PWS nucleic acids) can comprise, consist essentially of, or consist of the RNA sequence set forth in SEQ ID NO:4 with one, two, three, four, five, six, seven, eight, nine, or ten nucleotide deletions, additions, substitutions, or combinations thereof. In some cases, a SNORD 107 snoRNA that can be encoded by a nucleic acid encoding a SNORD107 snoRNA in an AAV vector provided herein (e.g., a single AAV vector designed to express one or more PWS nucleic acids) can comprise, consist essentially of, or consist of the RNA sequence set forth in SEQ ID NO:4 with two or less, three or less, four or less, five or less, six or less, seven or less, eight or less, nine or less, or ten or less nucleotide deletions, additions, substitutions, or combinations thereof.

In some cases, an AAV vector provided herein (e.g., an AAV vector designed to express one or more PWS nucleic acids) can include nucleic acid encoding a SNORD64 snoRNA. An AAV vector provided herein (e.g., an AAV vector designed to express one or more PWS nucleic acids) can include any appropriate nucleic acid encoding a SNORD64 snoRNA. For example, an AAV vector having a genome (e.g., a single-stranded or double-stranded DNA genome) including a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include any appropriate nucleotide sequence encoding a SNORD64 snoRNA.

A nucleic acid encoding a SNORD64 snoRNA in an AAV vector provided herein (e.g., a single AAV vector designed to express one or more PWS nucleic acids) can encode any appropriate SNORD64 snoRNA. For example, an AAV vector having a genome (e.g., a single-stranded or double-stranded DNA genome) including a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include nucleic acid encoding any appropriate SNORD64 snoRNA. In some cases, a SNORD64 snoRNA that can be encoded by a nucleic acid encoding a SNORD64 snoRNA in an AAV vector provided herein (e.g., an AAV vector designed to express one or more PWS nucleic acids) can have the nucleic acid sequence set forth in the NCBI databases at Accession No. NR_001294. In some cases, a SNORD64 snoRNA that can be encoded by a nucleic acid encoding a SNORD64 snoRNA in an AAV vector provided herein (e.g., an AAV vector designed to express one or more PWS nucleic acids) can have the RNA sequence set forth in SEQ ID NO:5. In some cases, a SNORD64 snoRNA that can be encoded by a nucleic acid encoding a SNORD64 snoRNA in an AAV vector provided herein (e.g., an AAV vector designed to express one or more PWS nucleic acids) can comprise, consist essentially of, or consist of the RNA sequence set forth in SEQ ID NO: 5. In some cases, a SNORD64 snoRNA that can be encoded by a nucleic acid encoding a SNORD64 snoRNA in an AAV vector provided herein (e.g., an AAV vector designed to express one or more PWS nucleic acids) can comprise, consist essentially of, or consist of the RNA sequence set forth in SEQ ID NO:5 with one, two, three, four, five, six, seven, eight, nine, or ten nucleotide deletions, additions, substitutions, or combinations thereof. In some cases, a SNORD64 snoRNA that can be encoded by a nucleic acid encoding a SNORD64 snoRNA in an AAV vector provided herein (e.g., a single AAV vector designed to express one or more PWS nucleic acids) can comprise, consist essentially of, or consist of the RNA sequence set forth in SEQ ID NO:5 with two or less, three or less, four or less, five or less, six or less, seven or less, eight or less, nine or less, or ten or less nucleotide deletions, additions, substitutions, or combinations thereof.

In some cases, an AAV vector provided herein (e.g., an AAV vector designed to express one or more PWS nucleic acids) can include nucleic acid encoding a SNORD116 snoRNA. An AAV vector provided herein (e.g., an AAV vector designed to express one or more PWS nucleic acids) can include any appropriate nucleic acid encoding a SNORD116 snoRNA. For example, an AAV vector having a genome (e.g., a single-stranded or double-stranded DNA genome) including a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include any appropriate nucleotide sequence encoding a SNORD116 snoRNA.

A nucleic acid encoding a SNORD116 snoRNA in an AAV vector provided herein (e.g., a single AAV vector designed to express one or more PWS nucleic acids) can encode any appropriate SNORD116 snoRNA. For example, an AAV vector having a genome (e.g., a single-stranded or double-stranded DNA genome) including a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include nucleic acid encoding any appropriate SNORD116 snoRNA. In some cases, a SNORD116 snoRNA that can be encoded by a nucleic acid encoding a SNORD116 snoRNA in an AAV vector provided herein (e.g., an AAV vector designed to express one or more PWS nucleic acids) can have the nucleic acid sequence set forth in the NCBI databases at Accession Nos. NR_003324 and NR_003332. In some cases, a SNORD116 snoRNA that can be encoded by a nucleic acid encoding a SNORD116 snoRNA in an AAV vector provided herein (e.g., an AAV vector designed to express one or more PWS nucleic acids) can have the RNA sequence set forth in SEQ ID NO:6 or SEQ ID NO: 7. In some cases, a SNORD116 snoRNA that can be encoded by a nucleic acid encoding a SNORD116 snoRNA in an AAV vector provided herein (e.g., an AAV vector designed to express one or more PWS nucleic acids) can comprise, consist essentially of, or consist of the RNA sequence set forth in SEQ ID NO:6 or SEQ ID NO:7. In some cases, a SNORD116 snoRNA that can be encoded by a nucleic acid encoding a SNORD116 snoRNA in an AAV vector provided herein (e.g., an AAV vector designed to express one or more PWS nucleic acids) can comprise, consist essentially of, or consist of the RNA sequence set forth in SEQ ID NO: 6 or SEQ ID NO:7 with one, two, three, four, five, six, seven, eight, nine, or ten nucleotide deletions, additions, substitutions, or combinations thereof. In some cases, a SNORD116 snoRNA that can be encoded by a nucleic acid encoding a SNORD116 snoRNA in an AAV vector provided herein (e.g., a single AAV vector designed to express one or more PWS nucleic acids) can comprise, consist essentially of, or consist of the RNA sequence set forth in SEQ ID NO:6 or SEQ ID NO:7 with two or less, three or less, four or less, five or less, six or less, seven or less, eight or less, nine or less, or ten or less nucleotide deletions, additions, substitutions, or combinations thereof.

In some cases, an AAV vector provided herein (e.g., an AAV vector designed to express one or more PWS nucleic acids) can include nucleic acid encoding a SNORD115 snoRNA. An AAV vector provided herein (e.g., an AAV vector designed to express one or more PWS nucleic acids) can include any appropriate nucleic acid encoding a SNORD115 snoRNA. For example, an AAV vector having a genome (e.g., a single-stranded or double-stranded DNA genome) including a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include any appropriate nucleotide sequence encoding a SNORD115 snoRNA.

A nucleic acid encoding a SNORD115 snoRNA in an AAV vector provided herein (e.g., a single AAV vector designed to express one or more PWS nucleic acids) can encode any appropriate SNORD115 snoRNA. For example, an AAV vector having a genome (e.g., a single-stranded or double-stranded DNA genome) including a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include nucleic acid encoding any appropriate SNORD115 snoRNA. In some cases, a SNORD115 snoRNA that can be encoded by a nucleic acid encoding a SNORD115 snoRNA in an AAV vector provided herein (e.g., an AAV vector designed to express one or more PWS nucleic acids) can have the nucleic acid sequence set forth in the NCBI databases at Accession No. NR_003304. In some cases, a SNORD115 snoRNA that can be encoded by a nucleic acid encoding a SNORD115 snoRNA in an AAV vector provided herein (e.g., an AAV vector designed to express one or more PWS nucleic acids) can have the RNA sequence set forth in SEQ ID NO:8. In some cases, a SNORD115 snoRNA that can be encoded by a nucleic acid encoding a SNORD115 snoRNA in an AAV vector provided herein (e.g., an AAV vector designed to express one or more PWS nucleic acids) can comprise, consist essentially of, or consist of the RNA sequence set forth in SEQ ID NO: 8. In some cases, a SNORD115 snoRNA that can be encoded by a nucleic acid encoding a SNORD115 snoRNA in an AAV vector provided herein (e.g., an AAV vector designed to express one or more PWS nucleic acids) can comprise, consist essentially of, or consist of the RNA sequence set forth in SEQ ID NO:8 with one, two, three, four, five, six, seven, eight, nine, or ten nucleotide deletions, additions, substitutions, or combinations thereof. In some cases, a SNORD115 snoRNA that can be encoded by a nucleic acid encoding a SNORD115 snoRNA in an AAV vector provided herein (e.g., a single AAV vector designed to express one or more PWS nucleic acids) can comprise, consist essentially of, or consist of the RNA sequence set forth in SEQ ID NO:8 with two or less, three or less, four or less, five or less, six or less, seven or less, eight or less, nine or less, or ten or less nucleotide deletions, additions, substitutions, or combinations thereof.

In some cases, an AAV vector provided herein (e.g., an AAV vector designed to express one or more PWS nucleic acids) can include nucleic acid encoding a SNORD109 snoRNA. An AAV vector provided herein (e.g., an AAV vector designed to express one or more PWS nucleic acids) can include any appropriate nucleic acid encoding a SNORD109 snoRNA. For example, an AAV vector having a genome (e.g., a single-stranded or double-stranded DNA genome) including a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include any appropriate nucleotide sequence encoding a SNORD109 snoRNA.

A nucleic acid encoding a SNORD109 snoRNA in an AAV vector provided herein (e.g., a single AAV vector designed to express one or more PWS nucleic acids) can encode any appropriate SNORD 109 snoRNA. For example, an AAV vector having a genome (e.g., a single-stranded or double-stranded DNA genome) including a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD 109 snoRNA can include nucleic acid encoding any appropriate SNORD109 snoRNA. In some cases, a SNORD 109 snoRNA that can be encoded by a nucleic acid encoding a SNORD109 snoRNA in an AAV vector provided herein (e.g., an AAV vector designed to express one or more PWS nucleic acids) can have the nucleic acid sequence set forth in the NCBI databases at Accession No. NR_001295. In some cases, a SNORD109 snoRNA that can be encoded by a nucleic acid encoding a SNORD109 snoRNA in an AAV vector provided herein (e.g., an AAV vector designed to express one or more PWS nucleic acids) can have the RNA sequence set forth in SEQ ID NO:9. In some cases, a SNORD109 snoRNA that can be encoded by a nucleic acid encoding a SNORD 109 snoRNA in an AAV vector provided herein (e.g., an AAV vector designed to express one or more PWS nucleic acids) can comprise, consist essentially of, or consist of the RNA sequence set forth in SEQ ID NO: 9. In some cases, a SNORD109 snoRNA that can be encoded by a nucleic acid encoding a SNORD109 snoRNA in an AAV vector provided herein (e.g., an AAV vector designed to express one or more PWS nucleic acids) can comprise, consist essentially of, or consist of the RNA sequence set forth in SEQ ID NO:9 with one, two, three, four, five, six, seven, eight, nine, or ten nucleotide deletions, additions, substitutions, or combinations thereof. In some cases, a SNORD 109 snoRNA that can be encoded by a nucleic acid encoding a SNORD109 snoRNA in an AAV vector provided herein (e.g., a single AAV vector designed to express one or more PWS nucleic acids) can comprise, consist essentially of, or consist of the RNA sequence set forth in SEQ ID NO:9 with two or less, three or less, four or less, five or less, six or less, seven or less, eight or less, nine or less, or ten or less nucleotide deletions, additions, substitutions, or combinations thereof.

In some cases, a DNA sequence including a nucleotide sequence encoding a NDN polypeptide and/or a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include one or more regulatory elements operably linked to the nucleotide sequence encoding a NDN polypeptide and/or to the nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA. For example, an AAV vector having a genome (e.g., a single-stranded or double-stranded DNA genome) including a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include one or more regulatory elements operably linked to the nucleotide sequence encoding a NDN polypeptide. For example, an AAV vector having a genome (e.g., a single-stranded or double-stranded DNA genome) including a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD 107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include one or more regulatory elements operably linked to the nucleotide sequence encoding a SNURF polypeptide, the nucleotide sequence encoding a SNRPN polypeptide, the nucleotide sequence encoding a SNORD107 snoRNA, the nucleotide sequence encoding a SNORD64 snoRNA, the nucleotide sequence encoding a SNORD116 snoRNA, the nucleotide sequence encoding a SNORD115 snoRNA, and the nucleotide sequence encoding a SNORD109 snoRNA. Such regulatory elements can include, without limitation, promoter sequences, enhancer sequences, response elements, signal peptides, internal ribosome entry sequences, polyadenylation signals, terminators, and inducible elements that modulate expression (e.g., transcription or translation) of a nucleic acid. The choice of regulatory element(s) that can be included in an AAV vector provided herein (e.g., an AAV vector designed to express one or more PWS nucleic acids) can depend on several factors, including, without limitation, inducibility, targeting, and the level of expression desired. For example, a promoter can be included in an AAV vector provided herein (e.g., an AAV vector designed to express one or more PWS nucleic acids) to facilitate transcription of a nucleic acid encoding a polypeptide, and/or a nucleotide sequence encoding a snoRNA. A promoter can be a naturally occurring promoter, a synthetic promoter, or a recombinant promoter. A promoter can be ubiquitous or inducible (e.g., in the presence of tetracycline), and can affect the expression of a nucleic acid encoding a polypeptide or a nucleic acid encoding a snoRNA in a general or tissue-specific manner.

The term “operably linked” as used herein with respect to a regulatory element and a nucleic acid encoding a polypeptide and/or a nucleic acid encoding a snoRNA refers to positioning of the regulatory element relative to the nucleic acid encoding a polypeptide and/or a snoRNA in such a way as to permit or facilitate expression of the polypeptide and/or snoRNA. For example, an AAV vector provided herein (e.g., an AAV vector designed to express one or more PWS nucleic acids) can contain a promoter and nucleic acid encoding a NDN polypeptide. In this case, the promoter can be operably linked to the nucleic acid encoding a NDN polypeptide such that it drives expression of the NDN polypeptide in cells.

In some cases, a promoter that can be used to drive expression of a polypeptide and/or a snoRNA in cells can include at least four (e.g., 4, 5, 6, 7, 8, 9, 10, or more) motifs having a DNA sequence of 5′-YGCGCANGCGCR-3′ (SEQ ID NO:10), each separated by an intervening nucleotide sequence. Examples of motifs having a DNA sequence of 5′-YGCGCANGCGCR-3′ (SEQ ID NO:10) are set forth in Table 1.

TABLE 1
Exemplary motifs having a DNA sequence
of 5′-YGCGCANGCGCR-3′ (SEQ ID NO: 10).

| DNA motif | SEQ ID NO |
|---|---|
| CGCGCATGCGCA | 11 |
| GTCGCATGCGCA | 12 |
| CGCGCAGGCGCG | 13 |
| TGCGCATGCGCG | 14 |
| TGCGCAAGCGCG | 15 |
| TGCGCATGCGCA | 16 |
| GGCGCCTGCGCA | 17 |
| CGCGCAGGCGCA | 18 |
| TGCGCAGGCGTG | 19 |
| GGCGCATGCGCA | 20 |

In some cases, a promoter that can be used to drive expression of a polypeptide and/or a snoRNA in cells can include 10 motifs having a DNA sequence of 5′-YGCGCANGCGCR-3′ (SEQ ID NO:10), with each adjacent motif being separated by an intervening nucleotide sequence. For example, a promoter that can be used to drive expression of a polypeptide and/or a snoRNA in cells can include motifs set forth in each of SEQ ID NOs: 11 to 20, with each adjacent motif being separated by an intervening nucleotide sequence. In some cases, a promoter that can be used to drive expression of a polypeptide and/or a snoRNA in cells can include 4 motifs having a DNA sequence of 5′-YGCGCANGCGCR-3′ (SEQ ID NO:10), with each adjacent motif being separated by an intervening nucleotide sequence. For example, a promoter that can be used to drive expression of a polypeptide and/or a snoRNA in cells can include motifs set forth in each of SEQ ID NOs: 11 to 14, with each adjacent motif being separated by an intervening nucleotide sequence.

A promoter that can be used to drive expression of a polypeptide and/or a snoRNA in cells can include at least four (e.g., 4, 5, 6, 7, 8, 9, 10, or more) motifs having a DNA sequence of 5′-YGCGCANGCGCR-3′ (SEQ ID NO:10), with each adjacent motif being separated by an intervening nucleotide sequence, can include any appropriate intervening nucleotide sequences. An intervening nucleotide sequence can include any appropriate number of nucleotides. In some cases, an intervening nucleotide sequence can include from about 8 to about 12 nucleotides. Examples of intervening nucleotide sequences that can be present between each adjacent motif having a DNA sequence of 5′-YGCGCANGCGCR-3′ (SEQ ID NO: 10) in a promoter that can be used to drive expression of a polypeptide and/or a snoRNA in cells are set forth in Table 2.

TABLE 2
Exemplary intervening nucleotide
sequence.

| DNA motif | SEQ ID NO |
|---|---|
| CCTCTGGGCC | 21 |
| CCAGTCTAGC | 22 |
| CTAGTCAGGC | 23 |
| CCATTGCCTG | 24 |
| CTACTCGCAC | 25 |
| CCTTTGCATG | 26 |
| CAATGGTCTG | 27 |
| CACGTCAGAC | 28 |
| CACGTCGGGC | 29 |

In some cases, a promoter that can be used to drive expression of a polypeptide and/or a snoRNA in cells can include 10 motifs having a DNA sequence of 5′-YGCGCANGCGCR-3′ (SEQ ID NO:10), with each adjacent motif being separated by an intervening nucleotide sequence. For example, a promoter that can be used to drive expression of a polypeptide and/or a snoRNA in cells can include 10 motifs having a DNA sequence of 5′-YGCGCANGCGCR-3′ (SEQ ID NO:10), with each adjacent motif being separated by an intervening nucleotide sequence set forth in each of SEQ ID NOs: 21 to 29. In some cases, a promoter that can be used to drive expression of a polypeptide and/or a snoRNA in cells can include 4 motifs having a DNA sequence of 5′-YGCGCANGCGCR-3′ (SEQ ID NO:10), with each adjacent motif being separated by an intervening nucleotide sequence. For example, a promoter that can be used to drive expression of a polypeptide and/or a snoRNA in cells can include 4 motifs having a DNA sequence of 5′-YGCGCANGCGCR-3′ (SEQ ID NO: 10), with each adjacent motif being separated by an intervening nucleotide sequence set forth in each of SEQ ID NOs: 21 to 23.

In some cases, a promoter that can be used to drive expression of a polypeptide and/or a snoRNA in cells can include the sequence set forth in SEQ ID NO:30.

In some cases, a promoter that can be used to drive expression of a polypeptide and/or a snoRNA in cells can include the sequence set forth in SEQ ID NO:31.

In some cases, a promoter that can be used to drive expression of a polypeptide and/or a snoRNA in cells can comprise, consist essentially of, or consist of one of the amino acid sequences set forth in Example 2. In some cases, a promoter that can be used to drive expression of a polypeptide and/or a snoRNA in cells can comprise, consist essentially of, or consist of one of the amino acid sequences set forth in Example 2 with one, two, three, four, five, six, seven, eight, nine, or ten amino acid or nucleotide deletions, additions, substitutions, or combinations thereof. In some cases, a promoter that can be used to drive expression of a polypeptide and/or a snoRNA in cells can comprise, consist essentially of, or consist of one of the amino acid sequences set forth in Example 2 with two or less, three or less, four or less, five or less, six or less, seven or less, eight or less, nine or less, or ten or less amino acid or nucleotide deletions, additions, substitutions, or combinations thereof.

In some cases, nucleotide sequences encoding snoRNAs (e.g., a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA) in a nucleic acid sequence (e.g., an engineered DNA sequence) in an AAV vector provided herein (e.g., an AAV vector designed to express one or more PWS nucleic acids) can be located between polypeptide-encoding portions (e.g., exons) of a nucleotide sequence encoding a SNURF polypeptide and/or polypeptide-encoding portions (e.g., exons) a nucleotide sequence encoding a SNRPN polypeptide. For example, a nucleic acid sequence (e.g., an engineered DNA sequence) including a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include (1) a first polypeptide-encoding portion of a nucleotide sequence encoding a SNURF polypeptide, followed by (2) a nucleotide sequence encoding a SNORD116 snoRNA, followed by (3) a second polypeptide-encoding portion of a nucleotide sequence encoding a SNURF polypeptide, followed by (4) a nucleotide sequence encoding a SNORD107 snoRNA, followed by (5) a third polypeptide-encoding portion of a nucleotide sequence encoding a SNURF polypeptide, followed by (6) a nucleotide sequence encoding a SNORD64 snoRNA, followed by (7) a first polypeptide-encoding portion of a nucleotide sequence encoding a SNRPN polypeptide, followed by (8) a nucleotide sequence encoding a SNORD109 snoRNA, followed by (9) a second polypeptide-encoding portion of a nucleotide sequence encoding a SNRPN polypeptide, followed by (10) a nucleotide sequence encoding a SNORD115 snoRNA, followed by 11) a third polypeptide-encoding portion of a nucleotide sequence encoding a SNRPN polypeptide.

In some cases, nucleotide sequences encoding snoRNAs (e.g., a nucleotide sequence encoding a SNORD 107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA) in a nucleic acid sequence (e.g., an engineered DNA sequence) in an AAV vector provided herein (e.g., an AAV vector designed to express one or more PWS nucleic acids) can be clustered within non-coding portions (e.g., introns) of a nucleotide sequence encoding a SNURF polypeptide and/or non-coding portions (e.g., introns) a nucleotide sequence encoding a SNRPN polypeptide. For example, a nucleic acid sequence (e.g., an engineered DNA sequence) including a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include (1) a nucleotide sequence encoding a SNURF polypeptide, followed by (2) a first polypeptide-encoding portion of a nucleotide sequence encoding a SNRPN polypeptide, followed by (3) a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA all clustered within a non-coding portion of a nucleotide sequence encoding a SNRPN polypeptide, followed by (4) a second polypeptide-encoding portion of a nucleotide sequence encoding a SNRPN polypeptide.

In some cases, nucleotide sequences encoding snoRNAs (e.g., a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA) in a nucleic acid sequence (e.g., an engineered DNA sequence) in an AAV vector provided herein (e.g., an AAV vector designed to express one or more PWS nucleic acids) can be located upstream (5′ of) and/or downstream (3′ of) a DNA sequence including a nucleotide sequence encoding a SNURF polypeptide and a nucleotide sequence encoding a SNRPN polypeptide. For example, a nucleic acid sequence (e.g., an engineered DNA sequence) including a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can include (1) a nucleotide sequence encoding a SNURF polypeptide, followed by (2) a first polypeptide-encoding portion of a nucleotide sequence encoding a SNRPN polypeptide, followed by (3) a second polypeptide-encoding portion of a nucleotide sequence encoding a SNRPN polypeptide, followed by (4) a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA all clustered within a non-coding portion of a nucleotide sequence encoding a SNRPN polypeptide located 3′ of the second polypeptide-encoding portion of a nucleotide sequence encoding a SNRPN polypeptide.

In some cases, a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can be flanked by a first ITR sequence and a second ITR sequence. In some cases, a first ITR sequence can be directly or indirectly followed by a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD 115 snoRNA, and a nucleotide sequence encoding a SNORD109 snoRNA. In some cases, a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA can be directly or indirectly followed by a second ITR sequence. Examples of ITR sequences that can flank a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD 109 snoRNA within an AAV vector provided herein are set forth in Table 3.

TABLE 3
Exemplary ITR sequences.

| | SEQ ID |
|---|---|
| DNA motif | NO |
| CCTGCAGGCAGCTGCGCGCTCGCTCGCTC | 32 |
| ACTGAGGCCGCCCGGGCAAAGCCCGGGCG | |
| TCGGGCGACCTTTGGTCGCCCGGCCTCAG | |
| TGAGCGAGCGAGCGCGCAGAGAGGGAGTG | |
| GCCAACTCCATCACTAGGGGTTCCT | |
| GGACGTCCGTCGACGCGCGAGCGAGCGAG | 33 |
| TGACTCCGGCGGGCCCGTTTCGGGCCCGC | |
| AGCCCGCTGGAAACCAGCGGGCCGGAGTC | |
| ACTCGCTCGCTCGCGCGTCTCTCCCTCAC | |
| CGGTTGAGGTAGTGATCCCCAAGGA | |
| AGGAACCCCTAGTGATGGAGTTGGCCACT | 34 |
| CCCTCTCTGCGCGCTCGCTCGCTCACTGA | |
| GGCCGGGCGACCAAAGGTTGCCCGACGCC | |
| CGGGCTTTGCCCGGGCGGCCTCAGTGAGC | |
| GAGCGAGCGCGCAGCTGCCTGCAGG | |
| TCCTTGGGGATCACTACCTCAACCGGTGA | 35 |
| GGGAGAGACGCGCGAGCGAGCGAGTGACT | |
| CCGGCCCGCTGGTTTCCAACGGGCTGCGG | |
| GCCCGAAACGGGCCCGCCGGAGTCACTCG | |
| CTCGCTCGCGCGTCGACGGACGTCC | |

In some cases, a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA within an AAV vector provided herein (e.g., an AAV vector designed to express one or more PWS nucleic acids) can be flanked by a first ITR sequence having a DNA sequence set forth in SEQ ID NO:32 and a second ITR sequence having DNA sequence set forth in SEQ ID NO:34.

In some cases, one or more AAV vectors provided herein (e.g., a population of a single AAV vector designed to express one or more PWS nucleic acids) can be formulated into a composition (e.g., a pharmaceutically acceptable composition). For example, one or more AAV vectors provided herein (e.g., a population of a single AAV vector designed to express one or more PWS nucleic acids) can be formulated together with one or more pharmaceutically acceptable carriers (additives), excipients, and/or diluents. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil.

In some cases, a composition containing one or more AAV vectors provided herein (e.g., a population of a single AAV vector designed to express one or more PWS nucleic acids) can be formulated for oral or parenteral (including, without limitation, intramuscular, intravenous, intradermal, intra-cerebral, intrathecal, or intraperitoneal (i.p.) injection) administration to the mammal. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

Also provided herein are nucleic acid molecules (e.g., isolated nucleic acid molecules) including a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA.

Also provided herein are cells (e.g., host cells) containing one or more AAV vectors provided herein (e.g., host cells containing a population of a single AAV vector designed to express one or more PWS nucleic acids). In some cases, a host cell containing one or more AAV vectors provided herein (e.g., a population of a single AAV vector designed to express one or more PWS nucleic acids) can be an in vitro cell (e.g., a cell in an in vitro culture).

Also provided herein are cells (e.g., host cells) containing a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD 109 snoRNA. In some cases, a host cell containing a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD 109 snoRNA can be an in vitro cell (e.g., a cell in an in vitro culture).

Examples of cells (e.g., host cells) that can contain one or more AAV vectors provided herein (e.g., a population of a single AAV vector designed to express one or more PWS nucleic acids) and/or a nucleic acid sequence (e.g., an engineered DNA sequence) including (a) a DNA sequence including a nucleotide sequence encoding a NDN polypeptide and (b) a DNA sequence including a nucleotide sequence encoding a SNURF polypeptide, a nucleotide sequence encoding a SNRPN polypeptide, a nucleotide sequence encoding a SNORD 107 snoRNA, a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA include, without limitation, epithelial cells such as HEK293 cells, endocrine cells, and neurons.

Also provided herein are methods for using one or more AAV vectors provided herein (e.g., a population of a single AAV vector designed to express one or more PWS nucleic acids). In some cases, one or more AAV vectors provided herein (e.g., a population of a single AAV vector designed to express one or more PWS nucleic acids) can be administered to a mammal (e.g., a human) having PWS to treat the mammal. For example, one or more AAV vectors provided herein (e.g., a population of a single AAV vector designed to express one or more PWS nucleic acids) can be used to increase expression of a NDN polypeptide, a SNURF polypeptide, a SNRPN polypeptide, a SNORD107 snoRNA, a SNORD64 snoRNA, a SNORD116 snoRNA, a SNORD115 snoRNA, and/or a SNORD109 snoRNA by cells within a mammal (e.g., a human) having PWS to treat the mammal.

In some cases, one or more AAV vectors provided herein (e.g., a population of a single AAV vector designed to express one or more PWS nucleic acids) can be administered to a mammal (e.g., a human) in need thereof (e.g., a human having PWS) to increase expression of a NDN polypeptide, a SNURF polypeptide, a SNRPN polypeptide, a SNORD 107 snoRNA, a SNORD64 snoRNA, a SNORD116 snoRNA, a SNORD115 snoRNA, and/or a SNORD109 snoRNA by cells within the mammal. In some cases, the materials and methods described herein can be used to increase expression of a NDN polypeptide, a SNURF polypeptide, a SNRPN polypeptide, a SNORD107 snoRNA, a SNORD64 snoRNA, a SNORD116 snoRNA, a SNORD115 snoRNA, and/or a SNORD109 snoRNA by cells within a mammal having PWS by, for example, 10, 20, 30, 40, 50, 60, 70, 80, 90, 95, or more percent.

One or more AAV vectors provided herein (e.g., a population of a single AAV vector designed to express one or more PWS nucleic acids) can increase expression of a NDN polypeptide, a SNURF polypeptide, a SNRPN polypeptide, a SNORD107 snoRNA, a SNORD64 snoRNA, a SNORD116 snoRNA, a SNORD115 snoRNA, and/or a SNORD109 snoRNA in any appropriate type of cells within a mammal (e.g., a human). In some cases, one or more AAV vectors designed to express one or more PWS nucleic acids can increase expression of a NDN polypeptide, a SNURF polypeptide, a SNRPN polypeptide, a SNORD 107 snoRNA, a SNORD64 snoRNA, a SNORD116 snoRNA, a SNORD115 snoRNA, and/or a SNORD109 snoRNA in pancreatic islet endocrine cells (e.g., pancreatic islet β-cells and pancreatic islet a-cells), gut endocrine cells, neurons (e.g., hypothalamic endocrine cells), pituitary neuroendocrine cells, or any combinations thereof.

In some cases, one or more AAV vectors provided herein (e.g., a population of a single AAV vector designed to express one or more PWS nucleic acids) can be administered to a mammal (e.g., a human) in need thereof (e.g., a human having PWS) to reduce or eliminate one or more symptoms of PWS. Examples of symptoms of PWS include, without limitation, hypotonia, distinct facial features (e.g., almond-shaped eyes, a narrowing of the head at the temples, a turned-down mouth, and a thin upper lip), poor infant sucking reflex, underdeveloped genitals, constant craving for food, rapid weight gain, short adult height, low muscle mass, high body fat, hypothyroidism, central adrenal insufficiency, intellectual disability, delayed motor development, speech problems (e.g., speech delays and poor articulation), scoliosis, hip problems, reduced saliva flow, nearsightedness, problems regulating body temperature, a high pain tolerance, hypopigmentation, hypoglycemia episodes, and hormone abnormalities (e.g., reductions in hormones such as growth hormone, insulin, pancreatic hormones, gut hormones, pituitary hormones, and/or hypothalamic hormones and increases in hormones such as ghrelin). In some cases, the materials and methods described herein can be used to reduce one or more symptoms of PWS within a mammal having PWS by, for example, 10, 20, 30, 40, 50, 60, 70, 80, 90, 95, or more percent.

Any appropriate mammal having PWS can be treated as described herein (e.g., by administering a population of a single AAV vector provided herein such as an AAV vector having genome including a nucleic acid sequence (e.g., an engineered DNA sequence) including (a) a nucleotide sequence including a nucleotide sequence encoding a NDN polypeptide, (b) a nucleotide sequence encoding a SNURF polypeptide, (c) a nucleotide sequence including a nucleotide sequence encoding a SNRPN polypeptide, (d) a nucleotide sequence encoding a SNORD107 snoRNA, (e) a nucleotide sequence encoding a SNORD64 snoRNA, (f) a nucleotide sequence encoding a SNORD116 snoRNA, (g) a nucleotide sequence encoding a SNORD115 snoRNA, and (h) a nucleotide sequence encoding a SNORD 109 snoRNA). Examples of mammals that can have PWS and that can be treated as described herein include, without limitation, humans, non-human primates (e.g., monkeys), dogs, cats, pigs, sheep, mice, and rats. In some cases, a human having PWS can be treated as described herein.

In some cases, the methods described herein can include identifying a mammal (e.g., a human) as having PWS by, for example, identifying that cells within the mammal include one or more mutations in an endogenous PWS locus. Any appropriate method can be used to identify the presence of one or more mutations in an endogenous PWS locus. For example, chromosomal analysis techniques can be used to identify the presence of one or more mutations in an endogenous PWS locus.

Any appropriate method can be used to deliver one or more AAV vectors provided herein (e.g., a population of a single AAV vector designed to express one or more PWS nucleic acids) can be administered to a mammal (e.g., a human) having PWS. In some cases, a composition (e.g., a pharmaceutically acceptable composition) including one or more AAV vectors provided herein (e.g., a population of a single AAV vector designed to express one or more PWS nucleic acids) can be administered locally or systemically. In some cases, a composition containing one or more AAV vectors provided herein (e.g., a population of a single AAV vector designed to express one or more PWS nucleic acids) can be administered locally via endoscopic retrograde cholangio-pancreatography (ERCP).

Any appropriate amount (e.g., any appropriate dose) of one or more AAV vectors provided herein (e.g., a population of a single AAV vector designed to express one or more PWS nucleic acids) can be administered to a mammal (e.g., a human) having PWS. An effective amount of a composition containing one or more AAV vectors provided herein (e.g., a population of a single AAV vector designed to express one or more PWS nucleic acids) can be any amount that can treat a mammal having PWS as described herein (e.g., that can increase expression of a NDN polypeptide, a SNURF polypeptide, a SNRPN polypeptide, a SNORD107 snoRNA, a SNORD64 snoRNA, a SNORD116 snoRNA, a SNORD115 snoRNA, and/or a SNORD109 snoRNA by cells within the mammal) without producing significant toxicity to the mammal. In some cases, an effective amount of one or more AAV vectors provided herein (e.g., a population of a single AAV vector designed to express one or more PWS nucleic acids) can be from about 1E+11 viral genomes to about 1E+13 viral genomes. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the PWS in the mammal being treated may require an increase or decrease in the actual effective amount administered.

One or more AAV vectors provided herein (e.g., a population of a single AAV vector designed to express one or more PWS nucleic acids) can be administered to a mammal (e.g., a human) having PWS in any appropriate frequency. The frequency of administration can be any frequency that can treat a mammal having PWS as described herein (e.g., that can increase expression of a NDN polypeptide, a SNURF polypeptide, a SNRPN polypeptide, a SNORD 107 snoRNA, a SNORD64 snoRNA, a SNORD116 snoRNA, a SNORD115 snoRNA, and/or a SNORD109 snoRNA by cells within the mammal) without producing significant toxicity to the mammal. For example, the frequency of administration can be from about once a day to about once a week, from about once a week to about once a month, or from about twice a month to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

One or more AAV vectors provided herein (e.g., a population of a single AAV vector designed to express one or more PWS nucleic acids) can be administered to a mammal (e.g., a human) having PWS for any appropriate duration. An effective duration for administering or using a composition containing one or more AAV vectors provided herein (e.g., a population of a single AAV vector designed to express one or more PWS nucleic acids) can be any duration that can treat a mammal having PWS as described herein (e.g., that can increase expression of a NDN polypeptide, a SNURF polypeptide, a SNRPN polypeptide, a SNORD 107 snoRNA, a SNORD64 snoRNA, a SNORD116 snoRNA, a SNORD115 snoRNA, and/or a SNORD109 snoRNA by cells within the mammal) without producing significant toxicity to the mammal. For example, the effective duration can vary from several weeks to several months, from several months to several years, or from several years to a lifetime. Multiple factors can influence the actual effective duration used for a particular treatment. For example, an effective duration can vary with the frequency of administration, effective amount, use of multiple treatment agents, and/or route of administration.

In some cases, methods for treating a mammal (e.g., a human) having PWS can include administering to the mammal one or more AAV vectors provided herein (e.g., a population of a single AAV vector designed to express one or more PWS nucleic acids) as the sole active ingredient to treat the mammal. For example, a composition containing one or more AAV vectors provided herein (e.g., a population of a single AAV vector designed to express one or more PWS nucleic acids) can include the one or more AAV vectors provided herein (e.g., a population of a single AAV vector designed to express one or more PWS nucleic acids) as the sole active ingredient in the composition that is effective to treat a mammal having PWS.

In some cases, methods for treating a mammal (e.g., a human) having PWS as described herein (e.g., by administering one or more AAV vectors provided herein such as AAV vectors designed to express one or more PWS nucleic acids) also can include administering to the mammal one or more (e.g., one, two, three, four, five or more) agents that can treat one or more symptoms of PWS. Examples of agents that can be used to treat one or more symptoms of PWS and can be administered together with one or more AAV vectors provided herein (e.g., a population of a single AAV vector designed to express one or more PWS nucleic acids) include, without limitation, GH treatments (e.g., human growth hormone (HGH) treatments), sex hormone treatments, psychiatric agents, and any combinations thereof. In some cases, agents that can be used to treat one or more symptoms of PWS can be as described elsewhere (see, e.g., Mahmoud et al., Int. J. Mol. Sci., 24(3): 2150 (2023); Miller et al., Diseases, 3(2): 78-85 (2015); and Tan et al., Obes. Rev., 21(5):e12992 (2020)). In cases where one or more AAV vectors provided herein (e.g., a population of a single AAV vector designed to express one or more PWS nucleic acids) are used in combination with one or more agents used to treat one or more symptoms of PWS, the one or more agents used to treat one or more symptoms of PWS can be administered at the same time (e.g., in a single composition containing both the one or more AAV vectors provided herein and the one or more agents used to treat one or more symptoms of PWS) or independently. For example, one or more AAV vectors provided herein (e.g., a population of a single AAV vector designed to express one or more PWS nucleic acids) can be administered first, and the one or more agents used to treat one or more symptoms of PWS administered second, or vice versa.

In some cases, methods for treating a mammal (e.g., a human) having PWS as described herein (e.g., by administering one or more AAV vectors provided herein such as AAV vectors designed to express one or more PWS nucleic acids) also can include subjecting the mammal one or more (e.g., one, two, three, four, five or more) additional therapies used to treat PWS. Examples of therapies that can be used to treat PWS include, without limitation, nutrition coaching (e.g., a healthy, reduced-calorie diet, a restricted-calorie diet, and supplemental vitamins and/or mineral), increasing physical activity and exercise, physical therapy (e.g., to improve movement skills and strength), speech therapy (e.g., to improve verbal skills), occupational therapy (e.g., to learn everyday skills), and combinations thereof. In cases where one or more AAV vectors provided herein (e.g., a population of a single AAV vector designed to express one or more PWS nucleic acids) are used in combination with one or more additional therapies used to treat PWS, the one or more additional therapies can be performed at the same time or independently of the administration of one or more AAV vectors provided herein (e.g., a population of a single AAV vector designed to express one or more PWS nucleic acids). For example, one or more AAV vectors provided herein (e.g., a population of a single AAV vector designed to express one or more PWS nucleic acids) can be administered before, during, or after the one or more additional therapies are performed.

The invention will be further described in the following examples, which do not limit the scope of the invention described in the claims.

### EXAMPLES

### Example 1: AAV Vectors with Miniaturized Promoters

This Example describes the design of AAV vectors that can express one or more PWS nucleic acids (e.g., AAV vectors designed to have a genome including a nucleic acid sequence (e.g., an engineered DNA sequence) including (a) a nucleotide sequence including a nucleotide sequence encoding a NDN polypeptide, (b) a nucleotide sequence encoding a SNURF polypeptide, (c) a nucleotide sequence including a nucleotide sequence encoding a SNRPN polypeptide, (d) a nucleotide sequence encoding a SNORD107 snoRNA, (e) a nucleotide sequence encoding a SNORD64 snoRNA, (f) a nucleotide sequence encoding a SNORD116 snoRNA, (g) a nucleotide sequence encoding a SNORD115 snoRNA, and (h) a nucleotide sequence encoding a SNORD109 snoRNA).

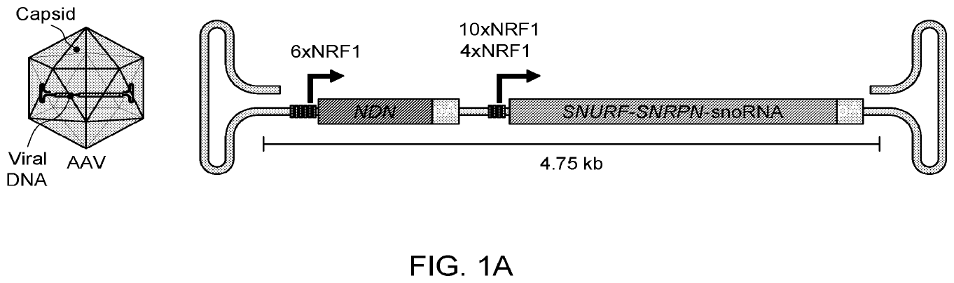

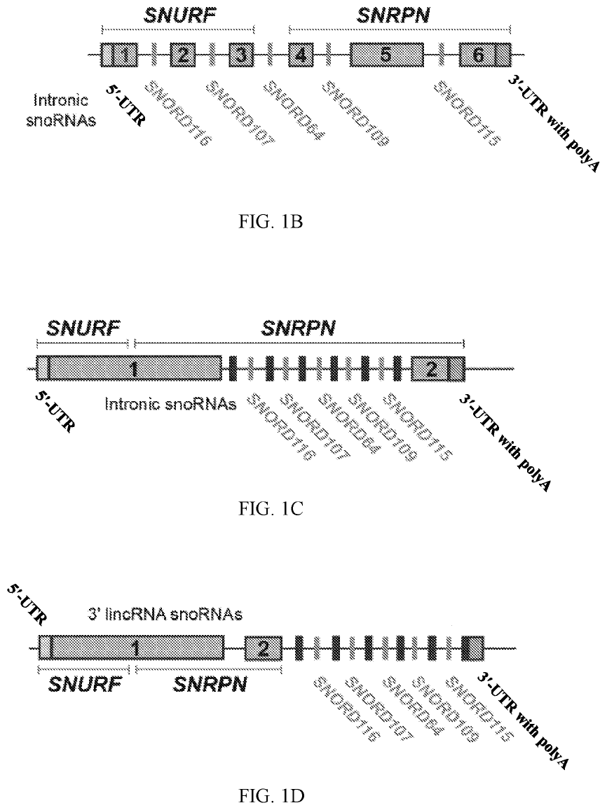

NRF1-array mini-promoters allow nucleic acid sequences that can encode 8 of the 10 major PWS genes to fit into a single AAV (FIG. 1A). Each vector insert includes SNURF (216-nt with stop codon; 71 amino acids) and SNRPN (723-nt; 240 amino acids) in the endogenous bicistronic layout with a 188-nt intercistronic region, as well as SNORD107, SNORD64, SNORD116, SNORD115, and SNORD109 snoRNAs (each 70-120 nt). For each of the duplicated SNORD116 and SNORD115 snoRNA families, a single “best matching sequence” was designed to maintain the conserved box C, box D, and putative antisense boxes for that specific snoRNA family. Three alternative vector inserts have been designed that include the nucleic acid encoding snoRNAs in the introns between protein-coding exons (FIG. 1B), clustered in SNURF-SNRPN introns (FIG. 1C), or located 3′ of SNURF-SNRPN (FIG. 1D). Additional vector design elements for each protein-coding transcription unit include a 5′-UTR (60-nt) and a 3′-UTR with a poly (A) motif (129-nt). AAV vectors containing inserts that contain nucleic acid that can encode one or more PWS nucleic acids are also referred to herein as AAV-PWS minigenes.

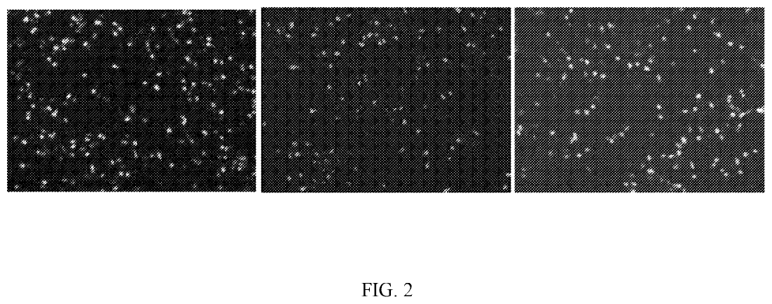

The CMV promoter-enhancer (613-nt) in an AAV vector was substituted with minimal synthetic NRF1-array mini-promoters. The 4×NRF1 (84-nt) and 10×NRF1 (216-nt) promoters were characterized, and it was shown that these promoters drive high levels of mNeonGreen expression in a variety of tested cell lines including INS1 β-cells (FIG. 2), fibroblasts, myoblasts, schwannoma, and others. Furthermore, these NRF1-array mini-promoters mimic endogenous regulation as in vivo expression of SNURF-SNRPN-snoRNA long intergenic non-coding RNAs (lincRNAs) is controlled by alternate 5′-promoters driven by several NRF1 binding sites and an intron 1 enhancer featuring NRF1. Likewise, in vivo expression of NDN is primarily regulated by NRF1.

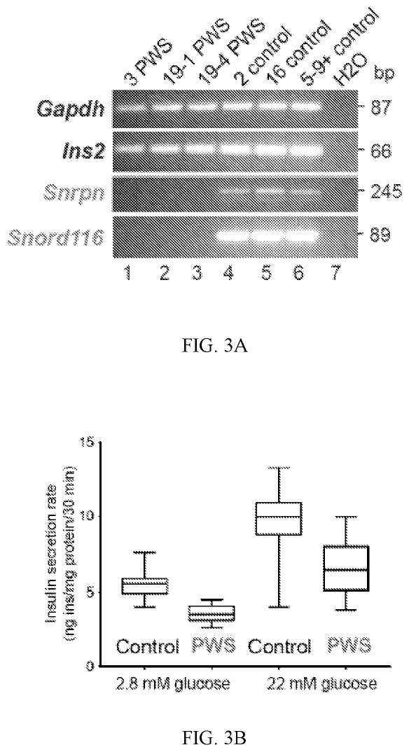

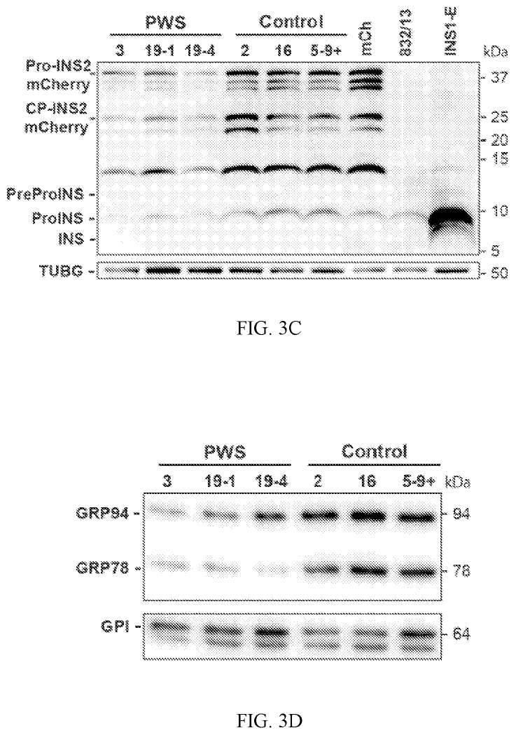

The ability of the AAV-PWS minigenes to treat PWS is tested in a cellular PWS model. PWS β-(INS-1) cell models show a cell-autonomous defect in insulin secretion along with deficits in RNA and protein production of multiple hormones and ER chaperones (FIG. 3A). AAV-PWS minigene vectors are packaged as serotype AAV6 or AAV8 for transduction of control and PWS-cell lines (3 each), and transduced cells were assessed for changes in levels of insulin secretion (FIG. 3B), mRNA levels for Ins1, Ins2, Iapp, Npy, and ER chaperones Sdf2l1, Dnajb11, Creld2, Grp78, Pdia4, Grp94, and Hyou1 using ddRT-PCR, and protein levels for peptide hormones (e.g., insulin) and major ER chaperones (FIG. 3C and FIG. 3D).

Designed vector inserts are produced by in vitro DNA synthesis and standard molecular cloning techniques used to insert between the AAV inverted terminal repeats (ITRs).

A multiplicity of infection (MOI) of 100,000 AAV genome copies/cell is used for cell transduction, with equivalent ddRT-PCR and protein (western blot) assays confirming the expression of each cistron.

mRNA (SNURF-SNRPN, NDN) and snoRNA expression is measured by droplet digital ddRT-PCR (with PCR primers specific for human exogenous vs. rat endogenous genes). Polypeptide expression (for SNURF, SmN/SNRPN, and NDN) is assessed by western blot analysis. Constructs that express all gene components are converted to AAV vectors.

### Example 2: Exemplary Promoter Sequences

### Exemplary 4×NRF1 Promoter Sequence

(SEQ ID NO: 30)
CGCGCATGCGCACCTCTGGGCCGTCGCATGCGCACCAGTCTAGCC
GCGCAGGCGCGCTAGTCAGGCTGCGCATGCGCG

### Exemplary 10×NRF1 Promoter Sequence

(SEQ ID NO: 31)
CGCGCATGCGCACCTCTGGGCCGTCGCATGCGCACCAGTCTAGCC
GCGCAGGCGCGCTAGTCAGGCTGCGCATGCGCGCCATTGCCTGTG
CGCAAGCGCGCTACTCGCACTGCGCATGCGCACCTTTGCATGGGC
GCCTGCGCACAATGGTCTGCGCGCAGGCGCACACGTCAGACTGCG
CAGGCGTGCACGTCGGGCGGCGCATGCGCA

### Example 3: Exemplary NDN Sequences

### Exemplary NDN Polypeptide Sequence

(SEQ ID NO: 1)

| | |
|---|---|
| 1 | MSEQSKDLSD PNFAAEAPNS EVHSSPGVSE |
| | GVPPSATLAE PQSPPLGPTA APQAAPPPQA |
| 61 | PNDEGDPKAL QQAAEEGRAH QAPSAAQPGP |
| | APPAPAQLVQ KAHELMWYVL VKDQKKMIIW |
| 121 | FPDMVKDVIG SYKKWCRSIL RRTSLILARV |
| | FGLHLRLTSL HTMEFALVKA LEPEELDRVA |
| 181 | LSNRMPMTGL LLMILSLIYV KGRGARESAV |
| | WNVLRILGLR PWKKHSTFGD VRKLITEEFV |
| 241 | QMNYLKYQRV PYVEPPEYEF FWGSRASREI |
| | TKMQIMEFLA RVFKKDPQAW PSRYREALEE |
| 301 | ARALREANPT AHYPRSSVSE D |

### Nucleic Acid Sequence Encoding SEQ ID NO:1

| (SEQ ID NO: 43) | |
|---|---|
| 1 | gcacttcctc tccaggaatc cgcggaggga gcgcaggctc gaagagctcc tggacgcaga |
| 61 | ggccctgccc ttgccagacg gcgcagacat gtcagaacaa agtaaggatc tgagcgaccc |
| 121 | taactttgca gccgaggccc ccaactccga ggtgcacagc agccctgggg tttcggaggg |
| 181 | ggttcctccg tccgcgaccc tggcagagcc gcagagccct cctctaggcc cgacggccgc |
| 241 | tccgcaggcc gcgccgcctc cccaggcccc gaacgacgag ggcgacccga aggccctgca |
| 301 | gcaggctgcg gaggagggcc gcgcccacca ggccccgagc gcggcccagc cgggcccggc |
| 361 | accgccagcc ccggcgcagc tggtgcagaa ggcgcacgag ctcatgtggt acgtgctggt |
| 421 | caaggaccag aagaagatga tcatctggtt tccagacatg gtgaaagatg tcatcggcag |
| 481 | ctacaagaag tggtgcagga gcatcctccg gcgcaccagc ctcatcctcg cccgggtgtt |
| 541 | cgggctgcac ctgaggctaa ccagcctgca caccatggag tttgcgctgg tcaaagcgct |
| 601 | ggagcccgag gagctggaca gggtggcgct gagcaaccgc atgcccatga caggcctcct |
| 661 | gctcatgatc ctgagcctca tctacgtgaa gggccgcggc gccagagaga gcgccgtctg |
| 721 | gaacgtgctg cgcatcctgg ggctgcggcc ctggaagaag cactccacct tcggggacgt |
| 781 | gcggaagctc atcactgagg agttcgtcca aatgaattac ctgaagtacc agcgcgtccc |
| 841 | atacgtggag ccgcccgaat acgagttctt ttggggctcc cgggccagcc gcgaaatcac |
| 901 | caagatgcaa atcatggagt tcctggccag ggtctttaag aaagaccccc aggcctggcc |
| 961 | ctcccgatac agagaagctc tggaggaggc cagagctctg cgggaggcta atcccactgc |
| 1021 | ccactaccct cgcagcagtg tctctgagga ctaccaaagt ctggaggcag atgaatggtt |
| 1081 | tctgaccctc accagggctg tggaagggtg ggggtgggtc attatagtat tcaggattta |
| 1141 | cagtgcagta ttcacgtgta acttttaagt tttcagtaca gtgcttttat acctttaatg |
| 1201 | caatgttgta ttcatttggg tactattgtg tagtatttag gatgtatgca tgtttgttta |
| 1261 | tatgtaagct tggttggtgc tttcgctttt gtgctacctt tcttggattt ttgtaccaga |
| 1321 | gatgtgctaa actgatgaaa tacattgaga aagtttccat cttattcttt tatatgggac |
| 1381 | tgatgatgtg tgttggggta gactgctcct gcagagtttg gaagaagtca ccagcaaagc |
| 1441 | cggcctaacc aagaaaagtc aaggcccttc atgaccttgc tgggcacaga aaacaccctc |
| 1501 | gtggagtaca ctaatttgaa ctggactggt ctcagtgtga gcacttggca cactttacta |
| 1561 | aacacatata caaccccacc gtgagtcaac tttaaagtaa acattaaaga ttcttgtgat |
| 1621 | acaatcattt ttggaaaagt gtactttatc attttaacaa agcagtatgg ttgggaatga |
| 1681 | gacaattctc tattttacag tgtatacaga tacaactatt tcccctaata gggtgggaaa |
| 1741 | aatcgctact catgattact cctaaatttg tgaagtttat agttctattg tctttaaatg |
| 1801 | taactcatgt ttatttcaaa aacattcaca aatatagaaa agtatacaaa acaaaacagt |
| 1861 | aagattgtct gtaatcacat catatgggaa taaaaaacaa aaataa |

first underlined section=start (ATG) codon
Second underlined section=stop (TAG) codon
third underlined section=polyA signal (AATAAA)

### Example 4: Exemplary SNURF Sequences

Exemplary SNURF polypeptide sequence

```
(SEQ ID NO: 2)
  1 MERARDRLHL RRTTEQHVPE VEVQVKRRRT
      ASLSNQECOL YPRRSQQQQV PVVDFQAELR
61 QAFLAETPRG G
```

### Nucleic Acid Sequence Encoding SEQ ID NO:2

| (SEQ ID NO: 36) | |
|---|---|
| 1 | gcagagtgga gcggccgccg gagatgcctg acgcatctgt ctgaggagcg gtcagtgacg |
| 61 | cgatggagcg ggcaagggat cgcttacacc tgagacgaac tacagaacag cacgtaccag |
| 121 | aggtggaagt ccaagtcaaa cgcagaagga ctgcctcact gagcaaccaa gagtgtcagt |
| 181 | tgtacccgag gcgttctcag cagcagcaag tacctgtggt ggatttccag gctgaactga |
| 241 | ggcaggcatt cttagctgag acaccaagag gtggttaaag ccatattgga gtagcgagga |
| 301 | atctgattcc aagcaaaaac caggctccat ctactctttg aagcttctgc ccagcttgca |
| 361 | ttgtttctag gagaacctgc gtcatacctt tatctatagc cttcccctag gtcttcagaa |
| 421 | gcatcaagtt ttaactgtgg acattggatt tggtggaaca gcaatcatga ctgttggcaa |
| 481 | gagtagcaag atgctgcagc acattgacta tagaatgaga tgtatcctgc aagatggccg |
| 541 | aatcttcatt ggcaccttta aggcttttga caagcatatg aatttgatcc tctgtgattg |
| 601 | tgatgagttc agaaagatca agccaaagaa tgcgaagcaa ccagagcgtg aagaaaagcg |
| 661 | ggttttgggt ctggtgttgc tgcgtgggga gaacttggta tccatgactg tggaggggcc |
| 721 | accccccaaa gatactggca ttgctcgggt accacttgct ggagctgctg gaggccctgg |
| 781 | ggttggtagg gcagctggta gaggagtacc agctggtgtg ccaattcccc aggcccctgc |
| 841 | tggattggca ggccctgtcc gaggagttgg gggaccatcc cagcaggtaa tgactccaca |
| 901 | gggaagaggc actgtagcag ctgctgctgt tgctgcgact gccagtattg ctggagcccc |
| 961 | aacacagtac ccaccaggac ggggcactcc gcccccaccc gtcggcagag caaccccacc |
| 1021 | tccaggcatt atggctcctc cacctggtat gagaccaccc atgggcccac caattgggct |
| 1081 | tccccctgct cgagggacgc caataggcat gccgcctccg ggaatgagac cccctccacc |
| 1141 | aggcattaga ggtccacctc ccccaggaat gcgtccacca agaccttagc atactgttga |
| 1201 | tccatctcag tcactttttc ccctgcaatg cgtcttgtga aattgtgtag agtgtttgtg |
| 1261 | agctttttgt tccctcattc tgcattaata atagctaata ataaatgcat agagcaatta |
| 1321 | aactgtgagg tactgttgta tatatttttt tgcctgttga ttttgatgag atcttaagtt |
| 1381 | actgtggatg agggtgatgc ctattaagca gttgattcaa atcatattct ctttaattct |
| 1441 | taggataaaa aggttttctg ctatctaa |

first underlined section=start (ATG) codon
Second underlined section=stop (TAG) codon
third underlined section=polyA signal (AATAAA)
fourth underlined section=polyA signal (ATTAAA)

### Example 5: Exemplary SNRPN Sequences

### Exemplary SNRPN Polypeptide Sequence

```
(SEQ ID NO: 3)
    1 MTVGKSSKML QHIDYRMRCI LQDGRIFIGT
        FKAFDKHMNL ILCDCDEFRK IKPKNAKQPE
  61 REEKRVLGLV LLRGENLVSM TVEGPPPKDT
        GIARVPLAGA AGGPGVGRAA GRGVPAGVPI
121 PQAPAGLAGP VRGVGGPSQQ VMTPQGRGTV
        AAAAVAATAS IAGAPTQYPP GRGTPPPPVG
181 RATPPPGIMA PPPGMRPPMG PPIGLPPARG
        TPIGMPPPGM RPPPPGIRGP PPPGMRPPRP
```

### Nucleic Acid Sequence Encoding SEQ ID NO:3

| (SEQ ID NO: 36) | |
|---|---|
| 1 | gcagagtgga gcggccgccg gagatgcctg acgcatctgt ctgaggagcg gtcagtgacg |
| 61 | cgatggagcg ggcaagggat cgcttacacc tgagacgaac tacagaacag cacgtaccag |
| 121 | aggtggaagt ccaagtcaaa cgcagaagga ctgcctcact gagcaaccaa gagtgtcagt |
| 181 | tgtacccgag gcgttctcag cagcagcaag tacctgtggt ggatttccag gctgaactga |
| 241 | ggcaggcatt cttagctgag acaccaagag gtggttaaag ccatattgga gtagcgagga |
| 301 | atctgattcc aagcaaaaac caggctccat ctactctttg aagcttctgc ccagcttgca |
| 361 | ttgtttctag gagaacctgc gtcatacctt tatctatagc cttcccctag gtcttcagaa |
| 421 | gcatcaagtt ttaactgtgg acattggatt tggtggaaca gcaatcatga ctgttggcaa |
| 481 | gagtagcaag atgctgcagc acattgacta tagaatgaga tgtatcctgc aagatggccg |
| 541 | aatcttcatt ggcaccttta aggcttttga caagcatatg aatttgatcc tctgtgattg |
| 601 | tgatgagttc agaaagatca agccaaagaa tgcgaagcaa ccagagcgtg aagaaaagcg |
| 661 | ggttttgggt ctggtgttgc tgcgtgggga gaacttggta tccatgactg tggaggggcc |
| 721 | accccccaaa gatactggca ttgctcgggt accacttgct ggagctgctg gaggccctgg |
| 781 | ggttggtagg gcagctggta gaggagtacc agctggtgtg ccaattcccc aggcccctgc |
| 841 | tggattggca ggccctgtcc gaggagttgg gggaccatcc cagcaggtaa tgactccaca |
| 901 | gggaagaggc actgtagcag ctgctgctgt tgctgcgact gccagtattg ctggagcccc |
| 961 | aacacagtac ccaccaggac ggggcactcc gcccccaccc gtcggcagag caaccccacc |
| 1021 | tccaggcatt atggctcctc cacctggtat gagaccaccc atgggcccac caattgggct |
| 1081 | tccccctgct cgagggacgc caataggcat gccgcctccg ggaatgagac cccctccacc |
| 1141 | aggcattaga ggtccacctc ccccaggaat gcgtccacca agaccttagc atactgttga |
| 1201 | tccatctcag tcactttttc ccctgcaatg cgtcttgtga aattgtgtag agtgtttgtg |
| 1261 | agctttttgt tccctcattc tgcattaata atagctaata ataaatgcat agagcaatta |
| 1321 | aactgtgagg tactgttgta tatatttttt tgcctgttga ttttgatgag atcttaagtt |
| 1381 | actgtggatg agggtgatgc ctattaagca gttgattcaa atcatattct ctttaattct |
| 1441 | taggataaaa aggttttctg ctatctaa |

first underlined section=start (ATG) codon
Second underlined section=stop (TAG) codon
third underlined section=polyA signal (AATAAA)
fourth underlined section=polyA signal (ATTAAA)

```
Example 6: Exemplary snoRNA Sequences
Exemplary SNORD107 snoRNA sequence
(SEQ ID NO: 4)
GCCUUGGCUAGGUUCAUGAUGACACAGGACCUUGUCUGAACAUAAUGAUUUCAAAAUUUGAGCUUAAAAAUGACACU
CUGAAAUCCAGUCAGUGU
Nucleic acid sequence encoding SEQ ID NO: 4
(SEQ ID NO: 37)
                            box C box D′ box C′ box D
GCCTTGGCTAGGTTCATGATGACACAGGACCTTGTCTGAACATAATGATTTCAAAATTTGAGCTTAAAAATGACACTCTGAAATCCAGTCAGTGT
Exemplary SNORD64 snoRNA sequence
(SEQ ID NO: 5)
GGGCUGGAUUUGUGAUGAGCUGUGUUUACUGAGCAUGAUGAAGUAAAGCUCAACGUGAUUACUCUGAAGUCCAGCCU
Nucleic acid sequence encoding SEQ ID NO: 5
(SEQ ID NO: 38)
                      box C box D′ box C′ box D
GGGCTGGATTTGTGATGAGCTGTGTTTACTGAGCATGATGAAGTAAAGCTCAACGTGATTACTCTGAAGTCCAGCCT
Exemplary SNORD116 snoRNA sequence
(SEQ ID NO: 6)
UGUGCUUGGAUCGAUGAUGAGUCCCCCAUAAAAACAUUCCUUGGAAAAGCUGAACAAAAUGAGUGAGAACUCAUACC
GUCGUUCUCAUCGGAACUGAGGUCCAGCACA
Nucleic acid sequence encoding SEQ ID NO: 6
(SEQ ID NO: 39)
                          box C box D′
TGTGCTTGGATCGATGATGAGTCCCCCATAAAAACATTCCTTGGAAAAGCTGAACAAA
  box C′ box D
ATGAGTGAGAACTCATACCGTCGTTCTCATCGGAACTGAGGTCCAGCACA
Exemplary SNORD116 snoRNA sequence
(SEQ ID NO: 7)
UGUGCAUGGAUCGAUGAUGACUUCCAUAUAUACAUUCCUUGGAAAGCUGAACAAAAUGAGUGAAAACUCUAUACCGU
CAUCCUCGUCGAACUGAGGUCCAGCACA
Nucleic acid sequence encoding SEQ ID NO: 7
(SEQ ID NO: 40)
                          box C box D′
TGTGCATGGATCGATGATGACTTCCATATATACATTCCTTGGAAAGCTGAACAAAA
  box C′ box D
TGAGTGAAAACTCTATACCGTCATCCTCGTCGAACTGAGGTCCAGCACA
Exemplary SNORD115 snoRNA sequence
(SEQ ID NO: 8)
UGCCCUGGGUUGGGUCAAUGAUGAGAACCUUAUAUUGUCCUGAAGAGAGGUGAUGACUUAAAAAUCAUGCUCAAUAG
GAUUACGCUGAGGCCCAGCCUAGGUG
Nucleic acid sequence encoding SEQ ID NO: 8
(SEQ ID NO: 41)
                                    box C box D′ box C′
TGCCCTGGGTTGGGTCAATGATGAGAACCTTATATTGTCCTGAAGAGAGGTGATGACTTAAAAATC
    antisense box box D
ATGCTCAATAGGATTACGCTGAGGCCCAGCCTAGGTG
Exemplary SNORD109 snoRNA sequence
(SEQ ID NO: 9)
UGCUUGUUUGGAUCGAUGAUGAGAAUAAUUGUCUGAGGAUGCUGAGGGACUCAUUCCAGAUGUCAAUCUGAGGUCCA
GAUGUGUG
Nucleic acid sequence encoding SEQ ID NO: 9
(SEQ ID NO: 42)
                                box C box D′ box C′ box D
TGCTTGTTTGGATCGATGATGAGAATAATTGTCTGAGGATGCTGAGGGACTCATTCCAGATGTCAATCTGAGGTCCAGATGTGTG
```

### Example 7: Treating PWS

A human identified as having PWS is administered a composition including a population of a single AAV vector designed to express one or more PWS nucleic acids (e.g., designed to have a genome including a nucleic acid sequence (e.g., an engineered DNA sequence) including (a) a nucleotide sequence including a nucleotide sequence encoding a NDN polypeptide, (b) a nucleotide sequence encoding a SNURF polypeptide, (c) a nucleotide sequence including a nucleotide sequence encoding a SNRPN polypeptide, (d) a nucleotide sequence encoding a SNORD107 snoRNA, (e) a nucleotide sequence encoding a SNORD64 snoRNA, (f) a nucleotide sequence encoding a SNORD116 snoRNA, (g) a nucleotide sequence encoding a SNORD115 snoRNA, and (h) a nucleotide sequence encoding a SNORD109 snoRNA). The single AAV vector designed to express one or more PWS nucleic acids is effective to increase expression of a NDN polypeptide, a SNURF polypeptide, a SNRPN polypeptide, a SNORD107 snoRNA, a SNORD64 snoRNA, a SNORD116 snoRNA, a SNORD115 snoRNA, and a SNORD109 snoRNA by cells within that mammal.

### Example 8: Treating PWS

A human identified as having PWS is administered a composition including a population of a single AAV vector designed to express one or more PWS nucleic acids (e.g., designed to have a genome including a nucleic acid sequence (e.g., an engineered DNA sequence) including (a) a nucleotide sequence including a nucleotide sequence encoding a NDN polypeptide, (b) a nucleotide sequence encoding a SNURF polypeptide, (c) a nucleotide sequence including a nucleotide sequence encoding a SNRPN polypeptide, (d) a nucleotide sequence encoding a SNORD107 snoRNA, (e) a nucleotide sequence encoding a SNORD64 snoRNA, (f) a nucleotide sequence encoding a SNORD116 snoRNA, (g) a nucleotide sequence encoding a SNORD115 snoRNA, and (h) a nucleotide sequence encoding a SNORD 109 snoRNA). The single AAV vector designed to express one or more PWS nucleic acids is effective to reduce one or more symptoms of PWS within that mammal.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An adeno-associated virus comprising a single-stranded DNA, wherein said single-stranded DNA comprises a first inverted terminal repeat (ITR) sequence followed by an engineered DNA sequence followed by a second ITR sequence, wherein said engineered DNA sequence comprises (a) a first DNA sequence comprising a first promotor sequence operably linked to first DNA segment comprising a nucleotide sequence encoding a necdin polypeptide and (b) a second DNA sequence comprising a second promotor sequence operably linked to a second DNA segment comprising a nucleotide sequence encoding a SNURF polypeptide, a nucleotide sequence encoding a SNRPN polypeptide, a nucleotide sequence encoding a SNORD107 small nucleolar RNA (snoRNA), a nucleotide sequence encoding a SNORD64 snoRNA, a nucleotide sequence encoding a SNORD116 snoRNA, a nucleotide sequence encoding a SNORD115 snoRNA, and a nucleotide sequence encoding a SNORD109 snoRNA, wherein each of said first promotor sequence and said second promotor sequence comprises at least four motifs having the DNA sequence set forth in SEQ ID NO: 10 (5′-YGCGCANGCGCR-3′), wherein 8 to 12 intervening nucleotides are located between each adjacent motif of said at least four motifs, and wherein delivery of said adeno-associated virus to a cell within said mammal results in expression of said necdin polypeptide, said SNURF polypeptide, said SNRPN polypeptide, said SNORD107 snoRNA, said SNORD64 snoRNA, said SNORD116 snoRNA, said SNORD115 snoRNA, and said SNORD109 snoRNA.

2. The adeno-associated virus of claim 1, said mammal is a human.

3-5. (canceled)

6. The adeno-associated virus of claim 1, wherein said adeno-associated virus is AAV6, AAV8, AAV3, AAVDJ, AAVKP1, or AAV9.

7. (canceled)

8. The adeno-associated virus of claim 1, wherein said first ITR sequence is indirectly followed by said engineered DNA sequence.

9. (canceled)

10. The adeno-associated virus of claim 1, wherein said engineered DNA sequence is indirectly followed by said second ITR sequence.

11. (canceled)

12. The adeno-associated virus of claim 1, wherein said first promotor sequence comprises four of said motifs.

13. The adeno-associated virus of claim 1, wherein said second promotor sequence comprises six of said motifs.

14. The adeno-associated virus of claim 1, wherein each of said motifs comprise a DNA sequence set forth in any one of SEQ ID NOs: 11 to 20.

15. The adeno-associated virus of claim 1, wherein each of said motifs comprise a DNA sequence set forth in any one of SEQ ID NOs: 11 to 14.

16-17. (canceled)

18. The adeno-associated virus of claim 1, wherein said SNURF polypeptide is a human SNURF polypeptide.

19. (canceled)

20. The adeno-associated virus of claim 1, wherein said SNRPN polypeptide is a human SNRPN polypeptide.

21. (canceled)

22. The adeno-associated virus of claim 1, wherein said SNORD107 snoRNA is a human SNORD107 snoRNA.

23. (canceled)

24. The adeno-associated virus of claim 1, wherein said SNORD64 snoRNA is a human SNORD64 snoRNA.

25. (canceled)

26. The adeno-associated virus of claim 1, wherein said SNORD116 snoRNA is a human SNORD116 snoRNA.

27-31. (canceled)

32. The adeno-associated virus of claim 1, wherein said first DNA segment comprises, downstream of said nucleotide sequence encoding said necdin polypeptide, a poly (A) signal.

33-34. (canceled)

35. The adeno-associated virus of claim 1, wherein said second DNA segment comprises, downstream of said nucleotide sequence encoding said SNURF polypeptide, said nucleotide sequence encoding said SNRPN polypeptide, said nucleotide sequence encoding said SNORD107 snoRNA, said nucleotide sequence encoding said SNORD64 snoRNA, said nucleotide sequence encoding said SNORD116 snoRNA, said nucleotide sequence encoding said SNORD115 snoRNA, and said nucleotide sequence encoding said SNORD109 snoRNA, a poly (A) signal.

36-42. (canceled)

43. A method for increasing expression of a necdin polypeptide, a SNURF polypeptide, a SNRPN polypeptide, a SNORD107 snoRNA, a SNORD64 snoRNA, a SNORD116 snoRNA, a SNORD115 snoRNA, and a SNORD109 snoRNA by cells within a mammal, wherein said method comprises administering an adeno-associated virus to said mammal, thereby increasing expression of said necdin polypeptide, said SNURF polypeptide, said SNRPN polypeptide, said SNORD107 snoRNA, said SNORD64 snoRNA, said SNORD116 snoRNA, said SNORD115 snoRNA, and said SNORD109 snoRNA by said cells within said mammal,
wherein said adeno-associated virus comprises a single-stranded DNA, wherein said single-stranded DNA comprises a first inverted terminal repeat (ITR) sequence followed by an engineered DNA sequence followed by a second ITR sequence, wherein said engineered DNA sequence comprises (a) a first DNA sequence comprising a first promotor sequence operably linked to first DNA segment comprising a nucleotide sequence encoding said necdin polypeptide and (b) a second DNA sequence comprising a second promotor sequence operably linked to a second DNA segment comprising a nucleotide sequence encoding said SNURF polypeptide, a nucleotide sequence encoding said SNRPN polypeptide, a nucleotide sequence encoding said SNORD107 snoRNA, a nucleotide sequence encoding said SNORD64 snoRNA, a nucleotide sequence encoding said SNORD116 snoRNA, a nucleotide sequence encoding said SNORD115 snoRNA, and a nucleotide sequence encoding said SNORD109 snoRNA, wherein each of said first promotor sequence and said second promotor sequence comprises at least four motifs having the DNA sequence set forth in SEQ ID NO:10 (5′-YGCGCANGCGCR-3′), wherein 8 to 12 intervening nucleotides are located between each adjacent motif of said at least four motifs.

44. The method of claim 43, wherein said mammal is a human.

45. A method for treating Prader-Willi syndrome, wherein said method comprises administering an adeno-associated virus of to a mammal having Prader-Willi syndrome, wherein expression of a necdin polypeptide, a SNURF polypeptide, a SNRPN polypeptide, a SNORD107 snoRNA, a SNORD64 snoRNA, a SNORD116 snoRNA, a SNORD115 snoRNA, and a SNORD109 snoRNA by cells within said mammal is increased following said administering step, and wherein severity of a symptom of said Prader-Willi syndrome is reduced following said administering step
wherein said adeno-associated virus comprises a single-stranded DNA, wherein said single-stranded DNA comprises a first inverted terminal repeat (ITR) sequence followed by an engineered DNA sequence followed by a second ITR sequence, wherein said engineered DNA sequence comprises (a) a first DNA sequence comprising a first promotor sequence operably linked to first DNA segment comprising a nucleotide sequence encoding said necdin polypeptide and (b) a second DNA sequence comprising a second promotor sequence operably linked to a second DNA segment comprising a nucleotide sequence encoding said SNURF polypeptide, a nucleotide sequence encoding said SNRPN polypeptide, a nucleotide sequence encoding said SNORD107 snoRNA, a nucleotide sequence encoding said SNORD64 snoRNA, a nucleotide sequence encoding said SNORD116 snoRNA, a nucleotide sequence encoding said SNORD115 snoRNA, and a nucleotide sequence encoding said SNORD109 snoRNA, wherein each of said first promotor sequence and said second promotor sequence comprises at least four motifs having the DNA sequence set forth in SEQ ID NO: 10 (5′-YGCGCANGCGCR-3′), wherein 8 to 12 intervening nucleotides are located between each adjacent motif of said at least four motifs.

46. The method of claim 45, wherein said mammal is a human.
